(19) United States
(12) Patent Application Publication
(10) Pub. No.: US 2007/0178586 A1
(43) Pub. Date: Aug. 2, 2007

(54) METHODS AND APPARATUSES FOR GROWING CELLS

(75) Inventors: Shang-Tian Yang, Dublin, OH (US); Anil Ouyang, Old Bridge, NJ (US)

Correspondence Address: CALFEE HALTER & GRISWOLD, LLP, 800 SUPERIOR AVENUE, SUITE 1400, CLEVELAND, OH, 44114, US

(73) Assignee: The Ohio State University Research Foundation, Columbus, OH (US)

(21) Appl. No.: 11/558,396

(22) Filed: Nov. 9, 2006

Related U.S. Application Data
(60) Provisional application No. 60/734,879, filed on Nov. 9, 2005.

(51) Int. Cl. C12N 5/06; C12M 3/00

(52) U.S. Cl. 435/325; 435/289.1

(57) ABSTRACT

Methods of culturing stem cells including growing fibroblast cells on a three-dimensional scaffold, perfusing the fibroblast cells with a cell culture medium to form fibroblast cell-conditioned cell culture medium, and growing the stem cells on a three-dimensional scaffold perfused with the fibroblast cell-conditioned cell culture medium are presented. Multi-stage bioreactors for growing stem cells, comprising a first fibrous bed bioreactor in fluid communication with a second fibrous bed bioreactor are also presented.

### CROSS REFERENCE TO RELATED APPLICATIONS

This application claims priority to and any other benefit of U.S. Provisional Application Ser. No. 60/734,879, filed on Nov. 9, 2005, the entire content of which is incorporated by reference herein.

### DESCRIPTION OF THE INVENTION

1. Field of the Invention

The methods and apparatuses generally relate to culturing of cells, particularly stem cells.

2. Background

Embryonic stem (ES) cells derived from the inner cell mass of blastocyst have unlimited proliferation potential and are totipotent. Embryonic germ (EG) cells originate from the reproductive cells of fetal cadaver tissue in the gonad ridge. EG cells are further along in development, and therefore cannot be derived from embryos, but instead must be isolated from fetal tissue. EG cells have the ability to form all three germ layers and therefore potentially all the organs of the body. Therefore, both are ideal cell sources for tissue engineering and cell therapy. Their vast potential clinical applications include treatments of diabetes, Parkinson's disease, spinal cord injury, liver malfunction, heart failure, and skin wounds. ES and EG cells are also invaluable tools for drug discovery and gene therapy. Genetically modified ES cells can be used for high-throughput drug screening and to transmit and express specific genes in target organs. Although the demand for ES and EG cells is high and expected to grow rapidly once their biomedical applications have been established, there has been little effort aimed at developing an economical method for mass production of ES and EG cells. Currently, the expansion of ES cells is based on common laboratory procedures carried out in two-dimensional (2-D) cell culture systems such as T-flasks, which are limited by the available surface area and difficult to scale up. Furthermore, the culture surface needs to be pre-coated with expensive extracellular matrix proteins, such as gelatin for murine (mES) and Matrigel for human (hES) ES cells. Frequent subculturing or passaging is also required in order to maintain the undifferentiated state of ES cells. These expensive, labor intensive, and time consuming 2-D culturing methods cannot meet the projected market demand for ES cells.

Recently, a three-dimensional (3-D) culturing method using fibrous matrices such as polyethylene terephthalate (PET) has been developed for culturing various cell types, including Chinese hamster ovary, hybridoma, osteosarcoma, cytotrophoblast, and mouse embryonic stem cells. In general, 3-D fibrous matrices can support high cell densities (3×10⁸ cells/mL matrix) and are used as tissue scaffolds because of their high porosity, specific surface area, permeability, and mechanical strength. In addition, the 3-D structure of fibrous matrices can provide cells a biomimetic environment that closely resembles their in vivo conditions. Cells cultured in the porous fibrous matrices are also protected from shear damage, a major concern in large-scale mammalian cell cultures. Such a 3-D culturing system is thus considered to be more scaleable and has been shown to be able to support and sustain mES cell growth and hematopoietic differentiation.

Current supplies of ES and EG cells are limited by the available cell sources, and there is a need to develop a scalable method for mass production of stem cells for biomedical applications. Current two-dimensional (2-D) culture systems are limited by space and low specific surface area available for cell adhesion and require coating the culture surfaces with expensive extracellular matrix proteins such as gelatin for mouse (mES) and Matrigel for human embryonic stem (hES) cells. This method also requires continuously subculturing ES cells every 2-3 days in media containing expensive growth factors in order to prevent undesirable spontaneous differentiation and to sustain cell proliferation and expansion to reach billions of cells needed for each application. In general, spontaneous differentiation occurs to embryonic stem cells cultured in vitro unless either a feeder layer of fibroblast cells or expensive growth factors are used in the ES cell culture. Using the feeder layer or the co-culture method generates a complication in their application due to the cell source contamination from the feeder layer cells when harvesting the ES cells. Therefore, a feeder free culture of hES cells on Matrigel coated surface was first developed by using mouse embryonic fibroblast (MEF) conditioned medium. Later research showed that basic fibroblast growth factors (bFGF) and other growth factors, such as TGF-β1, activin A, SCF, LIF, TPO, Flt3L, were also beneficial for the maintenance of hES cell's pluripotency in the absence of feeder layer cells. However, these growth factors are expensive and their use could impede the scale up of the ES cell production process.

There is a need for methods and devices to improve the expansion of embryonic stem cells and embryonic germ cells and their derivative cell lineages for cell therapy and other applications.

### SUMMARY OF THE INVENTION

The present methods and apparatuses, include, in various embodiments, two fibrous bed bioreactors in fluid communication wherein the first bioreactor is used to grow feeder cells which provide conditioned medium, and wherein the second bioreactor is used to grow cells of interest, such as stem cells. The conditioned medium is pumped from the first to the second bioreactor to support the growth of stem cells.

The various embodiments allow feeder cells to be separated from the stem cells, so as not to contaminate the final stem cell preparation. This physical separation also eliminates the needs of expensive cytokines and growth factors that are required for conventional methods of culturing stem cells.

The present methods and apparatuses, in various embodiments, provide methods of culturing stem cells comprising: growing the stem cells on a three-dimensional scaffold and perfusing the stem cells with culture medium. The perfusion may be continuous or intermittent.

In some embodiments, the three-dimensional scaffold comprises non-woven fibrous matrices. The non-woven fibrous matrices can be synthetic polymers such as polyethylene terepthalate or other polyester that exhibits an average pore size of less than or equal to about 150 μm, in some embodiments, from about 20 μm to about 150 μm, and in some embodiments, from about 30 μm to about 60 μm. In some embodiments, the three-dimensional scaffold lacks an ECM coating, wherein the ECM is chosen from gelatin, collagen, laminin, fibronectin, proteoglycan, entactin, heparin sulfate, Matrigel, and artificial ECM made of nanofibers.

In some embodiments, the feeder cells are fibroblasts such as STO and MEF (mouse embryonic fibroblasts) cells, and in some embodiments, the feeder cell-conditioned medium is prepared by perfusing fibroblast cells with cell culture medium, which may be conditioned. Some embodiments of the methods and apparatuses involve growing the fibroblast cells on a three-dimensional matrix. In some embodiments, the feeder cell culture medium lacks cytokine leukemia inhibitory factor (LIF) and other growth factors.

Some embodiments of the methods and apparatuses involve monitoring the culture medium for pH and/or degree of oxygenation. Some embodiments of the methods and apparatuses involve adjusting the pH and/or degree of oxygenation. Some embodiments of the methods and apparatuses include filtering the fibroblast cell-conditioned medium before perfusing the ES cells with the medium.

The methods and apparatuses further provide methods of culturing stem cells comprising: growing fibroblast feeder cells on a three-dimensional scaffold; perfusing the fibroblast cells with a cell culture medium to form fibroblast cell-conditioned cell culture medium; and growing the stem cells on a three-dimensional scaffold perfused with the fibroblast cell-conditioned cell culture medium. Some embodiments comprise filtering the fibroblast cell-conditioned medium before perfusing the stem cells with the medium. The stem cells may be harvested by steps that include contacting the stem cells with an enzyme chosen from accutase, trypsin and collagenase and/or increasing the perfusion flow rate over the stem cells.

The methods and apparatuses further provide methods of producing a differentiated cell culture, comprising culturing stem cells according to the methods presented herein, wherein the stem cells are co-cultured with cells of the differentiated cell type. The methods and apparatuses also provide methods of producing a differentiated cell culture, comprising culturing stem cells according to the methods presented herein, wherein the stem cells are perfused with culture medium comprising differentiated cell-conditioned culture medium.

The methods and apparatuses further provide multi-stage bioreactors for growing stem cells, comprising a first fibrous bed bioreactor in fluid communication with a second fibrous bed bioreactor. In some embodiments, the multi-stage bioreactor comprises a filter separating the first fibrous bed bioreactor from the second fibrous bed bioreactor, and in fluid connection with the first and second fibrous bed bioreactors. The multi-stage bioreactors may further comprise at least one tank in fluid connection with the second fibrous bed bioreactor. The multi-stage bioreactor may include a stem cell seeding tank, a stem cell harvesting tank, and/or a waste tank, any or all of which may be in fluid connection with the second fibrous bed bioreactor. In some embodiments, the multi-stage bioreactor includes a stem cell seeding tank, a stem cell harvesting tank, and a waste tank, each in fluid connection with the second fibrous bed bioreactor. The multi-stage bioreactors of the methods and apparatuses may further include at least one tank in fluid connection with the first fibrous bed bioreactor, which may be a culture medium reservoir. In some embodiments, the multi-stage bioreactors include at least one process control instrument for monitoring and/or adjusting oxygenation and/or pH of the first and/or second fibrous bed bioreactor.

Additional features and advantages of the methods and apparatuses will be set forth in part in the description which follows, and in part will be obvious from the description, or may be learned by practice of the methods and apparatuses. These features and advantages of the methods and apparatuses will be realized and attained by means of the elements and combinations particularly pointed out in the appended claims.

It is to be understood that both the foregoing general description and the following detailed description are exemplary and explanatory only and are not restrictive of the methods and apparatuses, as claimed.

The accompanying drawings, which are incorporated in and constitute a part of this specification, illustrate different embodiments of the methods and apparatuses and together with the description, serve to explain the principles of the methods and apparatuses.

### BRIEF DESCRIPTION OF THE DRAWINGS

FIG. 1. A two-stage perfusion ES cell culture system with the first bioreactor housing a feeder cell to condition the medium that is pumped through a filter and perfused to the second reactor housing ES cells is illustrated. Cells in both reactors are anchoraged in a 3-D fibrous matrix without pre-coating with ECM. After reaching a high cell density, the undifferentiated pluripotent ES cells can be harvested from the second reactor. In a similar scheme, the first reactor may house a selected somatic cell to condition the medium so as to induce the differentiation of ES cells to produce the same cell type used in the first reactor.

FIG. 2. Graphs showing a comparison of ES cell cultures on 2-D PET films and 3-D PET matrices with and without gelatin pre-coating. (A) ES cell growth expansion fold; (B) fraction of SSEA-1 positive cells.

FIG. 3. Graphs showing 2-D cultures of embryonic stem cells with 6 passages in the growth medium containing LIF, without LIF, and the conditioned medium (CM). (A) total cell number; (B) fraction of SSEA-1 positive cells.

FIG. 4. Graphs showing effects of initial lactic acid concentration in the growth media on ES cells in 2-D culture. (A) final cell number; (B) fractions of SSEA-1 and Oct-4 positive cells.

FIG. 5. Graphs showing 3-D cultures of embryonic stem cells in the growth medium containing LIF, without LIF, and the conditioned media (CM) at 10%, 25%, and 50% concentrations. (A) cell number, (B) fraction of Oct-4 positive cells; (C) fraction of SSEA-1 positive cells.

FIG. 6. Graphs showing effects of matrix pore size on ES cell cultures. (A) cell growth; (B) SSEA-1 expression; (C) Oct-4 expression.

FIG. 7. Scanning electron micrographs of ES cells in PET matrices. In general, large cell aggregates were formed in the large-pore matrix, while cells were more evenly distributed in the small-pore matrix. (A), (B) small-pore PET matrix; (C), (D) large-pore PET matrix. (Arrows pointing to the large cell aggregates)

FIG. 8. Graphs showing ES cell attachment kinetics in 3-D PET scaffold at 40 rpm, 80 rpm and 120 rpm

FIG. 9. SEM images of ES cells in PET scaffold (A, B) high cell density mES cells in PET scaffold on 15th day, (C, D) hES cells grow in 3-D spaces of the scaffold on 18^{th} day.

FIG. 10. Graphs showing a flow cytometry result of the marker expression (A) secondary antibody (IgG-FITC) control for Oct-4 measurement, (B) Oct-4 expression of mES cells in the reactor at 15^{th} day, (C) secondary antibody (IgM-PE) control for SSEA-1 measurement, (D) SSEA-1 expression of mES cells in the reactor at 15^{th} day, (E) secondary antibody (IgG-FITC) control for SSEA-4 measurement, (F) SSEA-4 expression of HES cells in the reactor at 18^{th} day.

FIG. 11. Graphs showing mathematical model predictions of (A) oxygen concentration at the outlet of the reactor and the total cell number in the fibrous bed bioreactor, (B) Predicted cell number in different size of fibrous bed bioreactor.

FIG. 12. Graphs showing neural differentiation of embryonic stem cells in various static culture systems within 7 days. (A) cell expansion fold (B) nestin expression. (Abbreviation: 2-D: cells grown on the surface in multiwells; 3-D: cells grown in the 3-D PET matrix in multiwells; DM: differentiation medium; CM: conditioned medium; RA: retinoic acid).

FIG. 13. Graphs showing Kinetics of neural differentiation from ES cells in different static culture systems. (A) 2-D culture (B) 3-D culture.

FIG. 14. Morphology of ES cells from neural differentiation in 2-D and 3-D cultures. (A) cells at the peripheral of the EB differentiated first on 2-D surface at 4^{th} day, (B) cells at the peripheral of the EB migrated and formed bridge with other EBs on 2-D surface at 6^{th} day, (C) neural network formation on 2-D surface at 15^{th} day, (D) neural network formation in 3-D PET scaffold at 10^{th} day, (E) differentiated ES cells on the PET fiber, (F) two neural cells on adjacent fibers communicate with each other by forming the neurite bridge.

FIG. 15. Graphs and micrograph showing neural differentiation of ES cells in the dynamic culture with microcarriers in a spinner flask agitated at 60 rpm. (A) Kinetics of cell growth, glucose consumption, and lactate production; (B) Cell populations expressing Oct-4, nestin, and NCAM, respectively; (C) Aggregates of ES cells and microcarriers.

FIG. 16. Graphs showing neural differentiation of ES cells in the dynamic culture with 3-D PET matrix in a spinner flask agitated at 60 rpm. (A) Kinetics of cell growth, glucose consumption, and lactate production; (B) Cell populations expressing Oct-4, nestin, and NCAM, respectively.

### DESCRIPTION OF THE EMBODIMENTS

Reference will now be made in detail to the present embodiments of the methods and apparatuses, examples of which are illustrated in the accompanying drawings. Wherever possible, the same reference numbers will be used throughout the drawings to refer to the same or like parts.

Unless otherwise defined, all technical and scientific terms used herein have the same meaning as commonly understood by one of ordinary skill in the art to which the methods and apparatuses belong. The terminology used in the description of the methods and apparatuses herein is for describing particular embodiments only and is not intended to be limiting of the methods and apparatuses. As used in the description of the methods and apparatuses and the appended claims, the singular forms “a,” “an,” and “the” are intended to include the plural forms as well, unless the context clearly indicates otherwise. All publications, patent applications, patents, and other references mentioned herein are expressly incorporated by reference in their entirety.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present methods and apparatuses. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the methods and apparatuses are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

The present methods and apparatuses specifically provide methods of culturing stem cells, the methods and apparatuses comprising: growing the stem cells on a three-dimensional scaffold in a first bioreactor and perfusing the stem cells with culture medium from a second bioreactor. The perfusion may be continuous or it may be intermittent.

In some embodiments, the cultured stem cells may comprise embryonic stem cells. In some embodiments, the embryonic stem cells may comprise human embryonic stem cells. In other embodiments, the cultured stem cells may comprise embryonic germ cells. In some embodiments, the embryonic germ cells may comprise human embryonic germ cells. In yet other embodiments, the cultured stem cells may comprise adult stem cells. In some embodiments, the adult stem cells may comprise human adult stem cells. In yet other embodiments, the cultured stem cells may comprise fetal stem cells. In some embodiments, the fetal stem cells may comprise human fetal stem cells.

In some embodiments, the three-dimensional scaffold comprises non-woven polyethylene terephthalate. In some embodiments, the three-dimensional scaffold comprises a non-woven polyester matrix. In other embodiments, the three-dimensional scaffold comprises a non-woven fibrous matrix. Other polymers can be used for the matrix, and those non-degradable polymers will be known to those of skill in the art. The non-woven polyethylene terepthalate or polyester matrix can exhibit an average pore size of less than or equal to about 150 μm, in some embodiments, from about 20 μm to about 150 μm, and in some embodiments, from about 30 μm to about 60 μm. Thus, the pore size can be less than or equal to 150, 140, 130, 120, 110, 100, 90, 80, 70, 60, 50, 40, 30, 20 microns, or even less. In some embodiments, the three-dimensional scaffold lacks components necessary for cell adhesion and growth. In some embodiments, the three-dimensional scaffold lacks an extracellular matrix (ECM) coating, wherein the ECM is chosen from gelatin, fibronectin, laminin, collagen, proteoglycan, entactin, heparin sulfate, Matrigel, and artificial ECM made of synthetic nanofibers. In some embodiments, the three-dimensional scaffold may comprise at least one ECM coating. In other embodiments, the three-dimensional scaffold may comprise at least two ECM coatings. In still other embodiments, the three-dimensional scaffold may comprise at least three ECM coatings.

The culture medium that is perfused from one bioreactor to another bioreactor comprise feeder cell-conditioned medium. In some embodiments, the feeder cells are fibroblast cells, and in some embodiments, the feeder cell-conditioned medium is prepared by perfusing fibroblast cells with cell culture medium. The feeder cell line can be any feeder cell line that can be used in expanding and/or supporting stem cells by the production of cytokines or growth factors. Some embodiments of the methods involve growing the fibroblast cells on a three-dimensional matrix. In some embodiments, the culture medium lacks added cytokine leukemia inhibitory factor; other normally required factors, which are known to those of skill in the art, may be omitted by practice of the methods and apparatuses presented herein.

Some embodiments of the methods and apparatuses involve monitoring the culture medium for pH and/or degree of oxygenation. Some embodiments of the methods and apparatuses involve adjusting the pH and/or degree of oxygenation. Some embodiments of the methods and apparatuses include filtering the fibroblast cell-conditioned medium before perfusing the stem cells with the medium.

The methods and apparatuses further provide methods of culturing stem cells comprising: growing fibroblast cells on a three-dimensional scaffold; perfusing the fibroblast cells with a cell culture medium to form fibroblast cell-conditioned cell culture medium; and growing the stem cells on a three-dimensional scaffold perfused with the fibroblast cell-conditioned cell culture medium. Some embodiments comprise filtering the fibroblast cell-conditioned medium before perfusing the stem cells with the medium. The stem cells may be harvested by steps that include contacting the stem cells with an enzyme chosen from ACCUTASE®, trypsin, collagenase, and/or increasing the perfusion flow rate over the ES cells. Other enzymes that may be used to loosen attached cells may be used as well, and are well known to those of skill in the art.

The methods and apparatuses further provide methods of producing a differentiated cell culture, comprising culturing stem cells according to the methods and apparatuses, wherein the stem cells are co-cultured with cells of the differentiated cell type. The methods and apparatuses also provide methods of producing a differentiated cell culture, comprising culturing stem cells according to the methods and apparatuses, wherein the stem cells are perfused with culture medium comprising differentiated cell-conditioned culture medium.

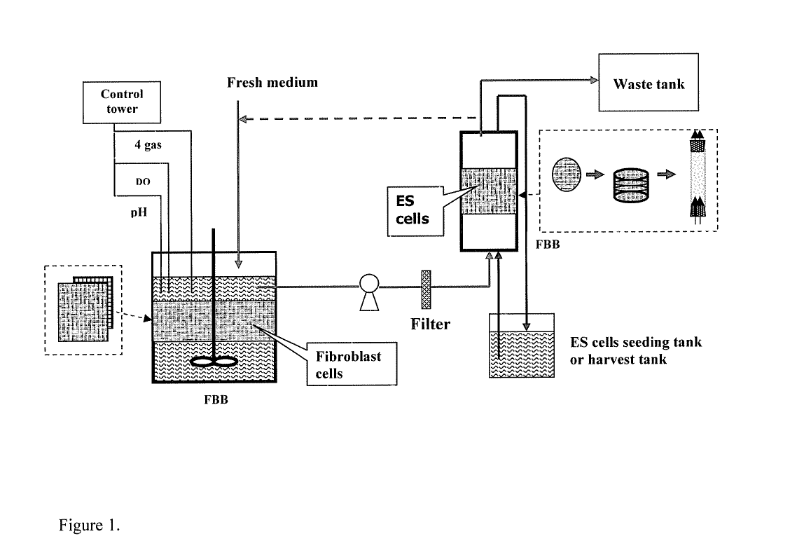

The methods and apparatuses further provide multi-stage bioreactors for growing stem cells, comprising a first fibrous bed bioreactor in fluid communication with a second fibrous bed bioreactor. In some embodiments, the multi-stage bioreactor comprises a membrane filter with a pore size of 0.2 μm to 0.45 μm separating the first fibrous bed bioreactor from the second fibrous bed bioreactor, and in fluid connection with the first and second fibrous bed bioreactors, as illustrated in FIG. 1. The filter prevents any feeder cells from the first reactor from being passed to the second reactor. The multi-stage bioreactors may further comprise at least one tank in fluid connection with the second fibrous bed bioreactor. The multi-stage bioreactor may include a stem cell seeding tank, a stem cell harvesting tank, and/or a waste tank, any of which may be in fluid connection with the second fibrous bed bioreactor. In some embodiments, the multi-stage bioreactor includes a stem cell seeding tank, a stem cell harvesting tank, and a waste tank, each in fluid connection with the second fibrous bed bioreactor. The multi-stage bioreactors of the methods and apparatuses may further include at least one tank in fluid connection with the first fibrous bed bioreactor, which may be a culture medium reservoir. In some embodiments, the multi-stage bioreactors include at least one process control instrument for monitoring and/or adjusting oxygenation and/or pH of the first and/or second fibrous bed bioreactor.

### EXAMPLE 1

### Two-Stage 3-D Bioreactor for Stem Cell Expansion

Materials and Methods

Cultures and Media

Murine ES D3 (mES) cells (CRL-1934, ATCC) were maintained on gelatin pre-coated T-flasks containing the ES growth medium, which consisted of the knock-out Dulbecco's Modified Eagle's Medium (DMEM) with 10% fetal bovine serum (FBS), 50 U/ml penicillin, 50 μg/ml streptomycin, 0.1 mM non-essential amino acids, 2 mM L-glutamine, 100 μM β-mercaptoethanol (Sigma, St. Louis, Mich.) and 100 μM leukemia inhibitory factor (LIF) (Chemicon, Temecula, Calif.). Both STO (CRL-1503, ATCC) and mouse embryonic fibroblast (MEF) (SCRC-1045, ATCC) cells were cultured in DMEM with 10% FBS, and they were used to prepare the conditioned media described later. Human ES (hES) cells (SCRC-2002, ATCC) were maintained on Matrigel (BD, San Jose, Calif.) coated T-flasks and cultured in the MEF conditioned medium described below supplemented with 4 ng/ml bFGF. Unless otherwise noted, these T-flask cultures were incubated in a CO₂ incubator at 37° C. The mES cells were passaged every three days and hES cells were sub-cultured every week to maintain their undifferentiated stage. All other cell culture materials were obtained from Gibco unless otherwise specified.

Conditioned Media

STO-Conditioned Media: To replace LIF in the ES growth medium, STO-conditioned media were prepared in either T-flasks or spinner flasks, as follows. After the STO fibroblast cell culture reached 80% confluence in 75 cm² T-flasks, the media were refreshed and cells were cultured for another four days. Supernatant was then collected and used as the conditioned medium after supplementing with glucose, non-essential amino acid, L-glutamine, β-mercaptoethanol, and antibiotics to the same concentrations as those in the ES growth medium described before. The conditioned medium used in the long-term mES culture was prepared in a 250-mL spinner flask by inoculating 3˜5×10⁶ STO cells to the flask containing PET matrix and 110 mL media. After incubation for six days, the medium was refreshed and cells were cultured for another four days before the supernatant was collected and supplemented with glucose, non-essential amino acid, L-glutamine, β-mercaptoethanol, and antibiotics to the same concentrations as those in the mES growth medium.

MEF-conditioned Medium: The conditioned medium for hES cell culture was prepared in a 250-mL spinner flask with the PET matrix seeded with ˜8 million MEF cells. The working volume is 100 ml. One day after inoculation of the MEF cells, the medium was changed to knock-out DMEM plus 10% serum replacement (SR), which was then collected after incubation for every three days and pooled in a big bottle. Prior to use, the conditioned medium was adjusted in its composition to: 4 g/L glucose, 10% SR, 0.1 mM non-essential amino acid, 2 mM L-glutamine, 100 μM β-mercaptoethanol, 50 U/mL penicillin, 50 μg/mL streptomycin.

Nonwoven PET Matrices

Needle-punched nonwoven polyethylene terephthalate (PET) matrices (thickness, 2 mm; porosity, 0.93; pore size: 60-130 μm; fiber diameter, ˜20 μm; fiber density, 1.35 g/cm³) were used as 3-D scaffolds. The PET matrices were pretreated with NaOH to increase their surface hydrophilicity. The thickness, porosity, and pore size of the PET matrices were reduced to ˜1 mm, 0.88, and 30˜60 μm, respectively, by thermal compression. In this paper, HP refers to the original PET matrices (without thermal compression) with the higher porosity, while LP refers to the matrices with the lower porosity obtained after thermal compression.

Static Cultures in Multiwells

The effects of gelatin coating, STO-conditioned media, lactic acid, and matrix pore size on ES cell proliferation and maintenance of its pluripotency were studied in static cultures. Unless otherwise noted, ES cells were inoculated into either gelatin-coated 24-well plates (2-D culture) or uncoated 3-D PET matrices in 24-well plates. For 3-D cultures, each well containing one sterile PET matrix and 100 μL medium was inoculated with 3˜10×10⁴ cells. The cell suspension was carefully added from the center of the matrix using a micropipette. Following three hours incubation in a CO₂ incubator to allow for cell attachment to the matrix, the seeded matrix was washed with the culture medium to remove unattached and loosely attached cells. Then, the matrix was transferred to a new well and 1 ml of fresh medium was added to the well.

Effects of Gelatin Coating

The necessity and effects of gelatin coating of the support surface on ES cells were studied. ES cells were cultured in multiwells each containing either a PET film disk (1.5 cm in diameter) or PET fibrous matrix (˜1 cm square). Both coated and uncoated PET films and matrices were used for direct comparison. For gelatin coating, the PET film or matrix was soaked in 0.3 ml of 0.5% gelatin solution for 2 h at room temperature, followed by air drying prior to use.

Effects of STO-Conditioned Medium

The feasibility of using STO-conditioned media to replace LIF for maintaining mES cells in the undifferentiated stage was studied. Each well was inoculated with 2×10⁴ cells and cultured in either the growth medium (with LIF or without LIF) or the STO-conditioned medium (without LIF). Cells were harvested every 2-3 days for analyses of total cell number and SSEA-1 positive cells. The harvested cells were sub-cultured in the same type of media for 6 consecutive passages, each time with the same inoculation amount of 2×10⁴ cells, to evaluate the long-term stability of ES cell culture maintained in different media.

Effects of Lactic Acid

The effects of lactic acid on ES cell proliferation and maintenance were also studied in 2-D static cultures. The growth media containing various initial amounts of lactic acid (0.162 to 3 g/L) were used to culture ES cells (2×10⁴) in gelatin-coated 24-well plates. The initial medium pH was adjusted with sodium bicarbonate to ˜7.0. After incubation for 4 days, cells were harvested to quantify the total cell number and SSEA-1 positive cells.

Effects of Matrix Pore Size

Long-term cultures in uncompressed (HP: 60˜130 μm pore size) and compressed (LP: 30˜60 μm pore size) PET matrices were compared to evaluate the effects of matrix pore size. About 3.5×10⁴ ES cells were inoculated into each well containing a piece of the PET matrix (1×1 cm) and 100 μL of the ES growth medium. Cells were harvested every 5 days for analyses of total cell number and SSEA-1 and Oct-4 positive cell populations.

Dynamic Cultures in Spinner Flasks

In order to avoid cell growth stagnation caused by oxygen limitation in static cultures, mES cells were cultured in a dynamic environment to increase oxygen transfer efficiency. The dynamic cultures were carried out in 25-mL spinner flasks, which were pre-siliconized with Sigmacote® (Sigma) to eliminate the possibility of cell attachment to the flask wall. The PET matrix was cut into 10 cm×1 cm sheet and affixed onto a stainless steel mesh around the wall of the spinner flask. After autoclaving at 121° C. for 30 min, 10 ml of cell suspension (5×10⁴ cells/mL) were added to the spinner flask. Unless otherwise noted, the spinner flasks were incubated in a CO₂ incubator at 37° C., agitated at 80 rpm, and with media change once every 2-3 days. Cell attachment kinetics in PET matrices was evaluated by counting the number of cells in the suspension during the first 2-5 h. The spinner cultures were studied for 15 days in five different media: the growth media with and without LIF, and the conditioned media of 15%, 25%, and 50% in the growth media without LIF. For hES cells culture, 0.3 million cells in 10 ml growth medium were added to a 25 ml spinner flask packed with 10 cm×1 cm PET scaffold. Medium was replenished every 2-3 days. Agitation rate is 80 rpm unless specified.

Perfusion Cultures in Controlled Bioreactor System

The two-stage perfusion bioreactor system set-up is illustrated in FIG. 1. A 450-mL spinner flask with the PET matrix (1×25×0.1 cm) was used as the first-stage bioreactor with its pH and DO probes connected to a New Brunswick Bioflow 30000 control system. Approximately 1×10⁷ STO cells were inoculated into this bioreactor, with 200 mL of working volume. The pH and DO were controlled at 7.0 and 70% air saturation, respectively, by continuously surface aeration of a gas mixture (air, CO₂, O₂, and N₂). After culturing the STO fibroblast cells for three days, ˜7.2×10⁶ mES cells in 4 mL were inoculated into the second-stage bioreactor, which was a glass column (2.5 cm diameter and 8 cm height) packed with PET matrices (2.5 cm diameter and 3 cm height). The entire bioreactor system was moved into a CO₂ incubator for the first 6˜8 h after mES inoculation. Then, it was re-connected to the control tower and medium circulation between the two bioreactors was started as 7.5 ml/min. The continuous perfusion was started 24 h after mES cell inoculation, at a dilution rate of 1 day⁻¹ or 8.3 ml/h. Liquid samples were taken every two days to monitor the concentrations of glucose, lactic acid, and suspended cells.

For the perfusion culture of hES cells, they were cultured in the 450-mL spinner flask reactor described above. Six million hES cells were inoculated into the bioreactor with 200 mL working volume and pH and DO controlled at 7.0 and 70% air saturation, respectively. The MEF conditioned medium with 4 ng/mL bFGF was perfused into the system from 7^{th} day at a perfusion rate of 1 day⁻¹.

At the end of the culture, cells were harvested from the PET scaffold. The culture medium was discarded and the reactor was filled with fresh PBS solution. Thereafter, the scaffold was washed by circulating PBS for 3 min. Following the washing, the PBS solution was removed from the vessel and 60 ml of Accutase® was pumped into the tank. The scaffold was immersed in the Accutase® for 20 minutes before being flushed. An additional 60 ml of Accutase® was circulated into the fibrous bed and incubated for another 20 minutes. The scaffold was then flushed and the total flushed solutions were subjected to centrifugation to collect the harvested cells.

Embryoid Body (EB) Forming Efficiency

To evaluate the pluripotency of the ES cells produced from various culturing systems, their EB forming efficiency was determined, as follows. Approximately one thousand ES cells were inoculated into a non-adhesive petri dish. After culturing for 6 days in the ES growth medium without LIF, the resulting EBs were counted. The EB forming efficiency was defined as the number of EB divided by the number of inoculated cells.

Flow Cytometric Analyses

ES cells were trypsinized from the PET matrices before flow cytometric analyses. For SSEA-1 assay, 2-5×10⁵ cells were washed with PBS containing 0.5% BSA and 2 mM EDTA (Sigma). Human FcR blocking reagent (Pharmigen, San Jose, Calif.) was added to the single cell suspension to prevent nonspecific binding; this was followed by incubation of SSEA-1 antibody (Developmental studies hybridoma bank, Iowa city, 10) for 60 min. at 4° C. After washing, the samples were incubated with goat anti-mouse IgM-PE secondary antibody (Jackson ImmunoResearch lab, West Grove, Pa.) for 30 min. After fixing the cells with 2% formaldehyde, the SSEA-1 positive cell population was analyzed using a flow cytometer (BD FACS Calibur). For intracellular protein Oct-4 quantification, cells were fixed with 4% formaldehyde for 20 min. at room temperature, followed by cell membrane perforation with the washing buffer containing 0.5% saponin and 0.1% sodium azide (Sigma) for 20 min at room temperature. The samples were later incubated with anti-Oct-4 monoclonal antibody (Chemicon, Temecula, Calif.) at room temperature for 30 min. and then IgG-FITC for another 30 min. The Oct-4 positive cells were then analyzed and quantified using a flow cytometer. SSEA-4 assay for hES cells was similar to SSEA-1 assay except that IgG-FITC, instead of IgM-PE, was used as the secondary antibody. Unstained cells were used for locating the population and cells only labeled with IgM-PE or IgG-FITC were used to evaluate the non-specific binding or background fluorescence reading. 10,000 gated cells were tested for markers measurement.

Scanning Electron Microscopy (SEM)

Cell morphology and distribution in the PET matrices were observed using a scanning electron microscope (Philip XL 30, Philips Electronics, Eindhoven, The Netherlands). Each matrix sample with cells was washed with PBS solution and incubated in 2.5% (v/v) glutaraldehyde at 4° C. overnight. The samples were then rinsed with distilled water and progressively dehydrated in 10% (v/v)-100% (v/v) ethanol in the increment of 10% by soaking the sample for 30 min. at each ethanol concentration. The dehydrated samples were then dried by soaking in hexamethyldisilazane (HMDS) (Sigma) and ethanol mixture with ascending HMDS concentrations of 1:3, 1:1, and 3:1 for final dehydration. The dried samples were sputter-coated with gold-palladium at an argon pressure of 14 Pa for 120 seconds and a current of 17 mA to convey electrical conductivity, and then observed in the SEM at 5-25 kV accelerating voltage.

Analytical Methods

Cell Counting. The PET matrices were placed into the standard nuclei counting solution (0.1 M citric acid, 0.1% (w/v) crystal violet) and incubated at 37° C. for 24 h. The matrix was then vigorously vortexed to release cell nuclei, which were counted under a microscope. Cells on 2-D surface of multiwells or T-flasks were counted, after trypsinization, using a hemocytometer.

Metabolic Assays. Lactic acid and glucose concentrations in the culture media were measured with YSI Biochemistry Select Analyzer (Yellow Springs, Ohio).

Results

Effects of Gelatin Coating

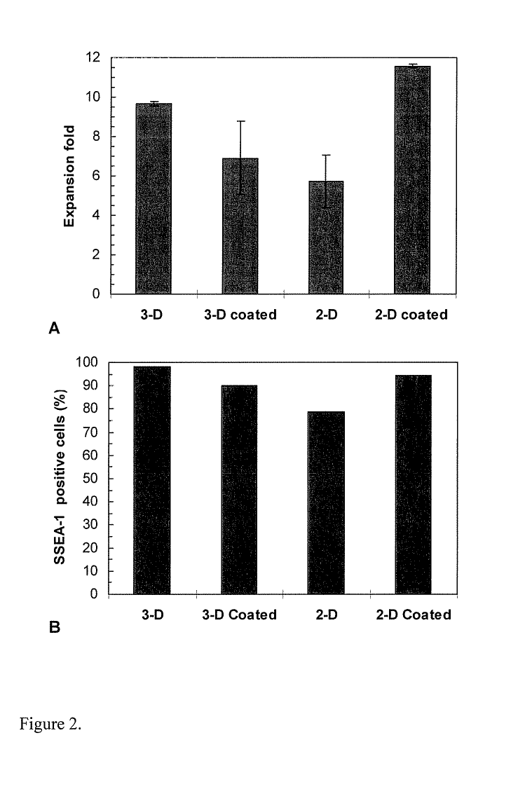

FIG. 2 compares the results of ES cells cultured in 2-D and 3-D with or without surface coating with gelatin. In 2-D cultures, cells generally grew better on the gelatin-coated surface than on the uncoated surface, as indicated by the higher expansion fold and fraction of SSEA-1 positive cells. This finding is consistent with common belief that gelatin or ECM protein coating on the support surface is indispensable for cell attachment and growth in ES cell cultures. However, 3-D cultures with PET scaffolds without gelatin pre-coating were able to support good cell growth and maintain high SSEA-1 expression (see FIG. 2). It is noted that gelatin coating of the PET matrices resulted in poorer cell growth because the uneven coating had blocked and deteriorated the 3-D pore structure of the matrices. Eliminating the expensive ECM coating of the support surface would be an advantage of 3-D cultures over 2-D cultures because it can significantly reduce the cost of the ES cell expansion process. This 3-D culturing advantage was also demonstrated with human ES cells cultured in PET scaffolds without coating with Matrigel or any ECM proteins as usually required in 2-D cultures. In addition, ES cells harvested from the 3-D culture showed a comparable or even higher EB forming efficiency (24.7%±7.2% vs. 18.7%±7.6% in 2-D cultures). The EB forming efficiency is an indicative of the pluripotency of ES cells or their ability to differentiate into various cell types. Therefore, ES cells grown in 3-D PET matrices can be maintained in their undifferentiated stage to conserve their pluripotency.

Effects of Conditioned Media

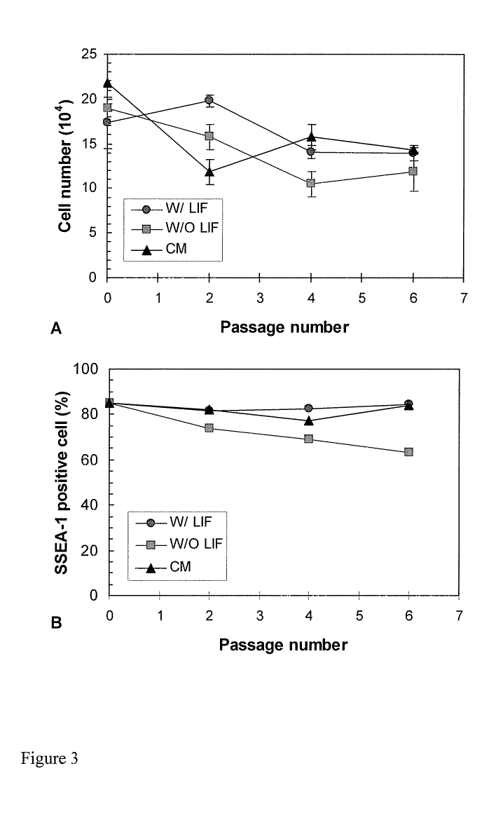

STO cells are commonly used as feeder cells to support the undifferentiated mES cell growth. These cells secrete several cytokines that are essential for mES cells to maintain their undifferentiated state. The conditioned medium from STO cell culture was thus studied as an economic alternative to the more expensive cytokine LIF commonly used in mES cell growth media. As shown in FIG. 3, no significant difference in cell growth, as indicated by the total cell number at the end of each passage, was found among the three media studied in 2-D cultures. However, SSEA-1 was significantly down-regulated in mES cells cultured in the growth medium without LIF, whereas cells cultured in the conditioned medium and the LIF-containing growth medium maintained their high SSEA-1 expression level, ˜85%. The results suggested that the STO-conditioned medium was as good as the more expensive LIF-containing growth media in maintaining mES cells in the undifferentiated stage.

Effects of Lactic Acid

However, it was later found that the conditioned media produced in the spinner flask could not sustain ES cell growth and maintain their undifferentiated stage in long-term 3-D cultures. The fraction of SSEA-1 positive cells dropped from 98% to 58% in the 3-D culture after 20 days. Further investigation revealed that the lactic acid production in the 3-D STO culture was much higher than that in 2-D STO cultures, resulting in the higher lactic acid concentration of the conditioned media from the 3-D culture (2.2-2.4 g/L vs. 0.6-0.9 g/L from the 2-D culture). The higher density of STO cells in the 3-D culture resulted in limited oxygen transfer and thus higher lactic acid production.

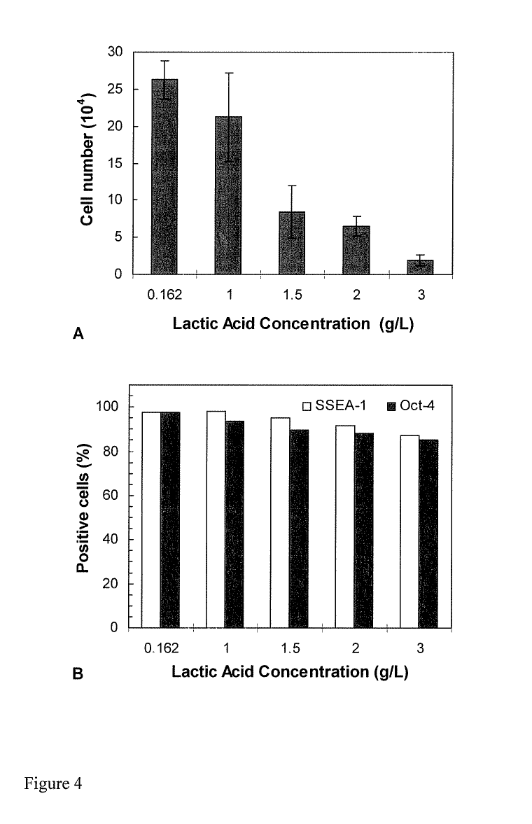

Since lactic acid is a known inhibitor to most mammalian cells, it was believed to be the main factor limiting mES cells growth in the conditioned media with a high lactic acid content. The effects of lactic acid on mES cells were thus studied and the results are shown in FIG. 4. As expected, lactic acid strongly inhibited ES cell growth at the concentrations of 1.5 g/L and higher. At 3 g/L of lactic acid, it was observed that mES cells could not attach well on the flask surface and there was no significant cell growth. Moreover, increasing the lactic acid concentration also significantly decreased the cell populations expressing Oct-4 and SSEA-1, implying that mES cells had undergone spontaneous differentiation at elevated lactic acid concentrations. It is clear that ES cells are more sensitive to lactic acid than other animal cells, which usually can tolerate lactic acid up to 3.5 g/L. This result concurred with the finding that frequent media replacement to minimize the accumulation of metabolic wastes was necessary for long-term ES cell cultures.

Long-Term 3-D Cultures in Conditioned Media

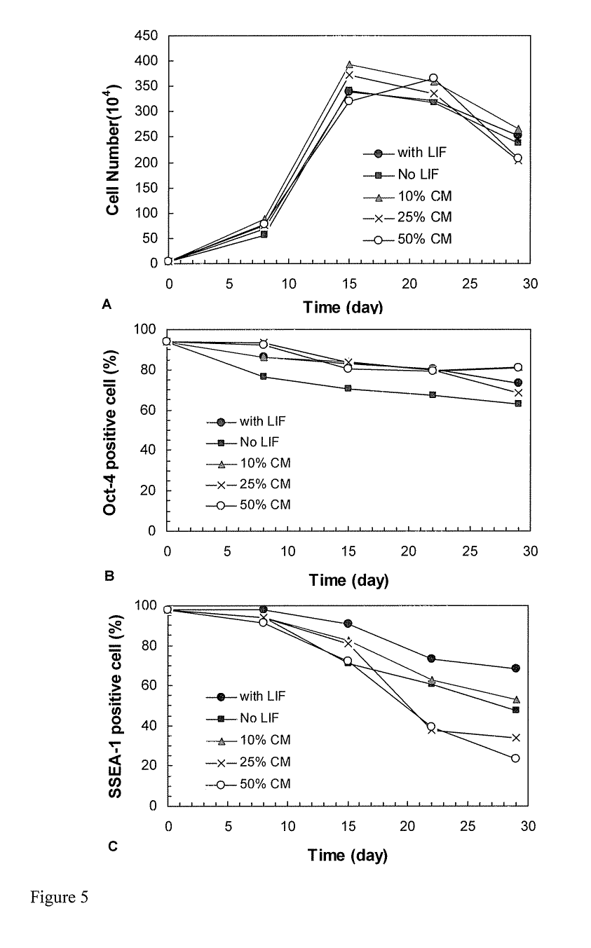

The conditioned medium was mixed with fresh ES growth medium (without LIF) at different ratios to reduce the amount of lactic acid present in the resulting media, which were used to test their effects on the long-term 3-D culture of mES cell. The results are shown in FIG. 5. In general, ES cells grown in these media showed similar growth kinetics. Except for the 50% conditioned medium, ES cells had expanded approximately 80-98 times and reached their maximum numbers after 15 days (FIG. 5A). As expected, the fraction of cells expressing Oct-4 decreased significantly faster in the culture without LIF than with LIF or the conditioned media (FIG. 5B). However, all cultures showed significant decrease in SSEA-1 positive cell population after 15 days (FIG. 5C), which marked the beginning of the stationary phase when cell growth was limited by oxygen and a dramatic drop in SSEA-1 expression occurred. The effect of oxygen starvation in the 3-D culture will be discussed later.

SSEA-1 is a surface marker and thus more sensitive to the culture environment than the intracellular protein Oct-4. It is noted, however, that SSEA-1 expression decreased much faster in 25% and 50% conditioned media, in which the lactic acid concentrations increased above the inhibiting level of 1.5 g/L (data not shown). It is thus clear that a high lactic acid concentration not only inhibited cell growth but also induced spontaneous differentiation. In general, the 10% conditioned medium was able to replace the LIF-containing medium for the long-term 3-D mES culture. As can be seen in Table 1, for the first 15 days ES cell growth in the 10% conditioned medium was 16% higher and Oct-4 and SSEA-1 levels were within the range as compared to the LIF-containing ES growth medium. Therefore, undifferentiated ES cells can be expanded and maintained in STO-conditioned media provided that the lactic acid concentration is properly controlled at a low level. This conclusion was confirmed with a dynamic ES culture carried out with a perfusion bioreactor where oxygen and lactate concentrations were controlled at proper levels.

TABLE 1
mES cell culture performance after 15 days of static culturing in various media.

| | With LIF | No LIF | 10% CM | 25% CM | 50% CM |
|---|---|---|---|---|---|
| Expansion fold* | 90.5 ± 3.4 | 91.3 ± 7.7 | 105.2 ± 11.1 | 99.1 ± 16.2 | 85.3 ± 12.5 |
| Oct-4 (%) | 83.2 | 70.9 | 84.3 | 83.8 | 80.4 |
| SSEA-1 (%) | 90.7 | 71.0 | 82.3 | 80.7 | 72.1 |
| Y_{lac/glu} (g/g) | 0.88 ± 0.04 | 0.89 ± 0.06 | 0.88 ± 0.1 | 0.85 ± 0.11 | 0.84 ± 0.09 |

*Initial cell number was 3.75 × 10⁴ cells/well
Abbreviation: CM, conditioned mediuml Y_{lac/glu}, lactic acid yield from glucose

Effects of Matrix Pore Size

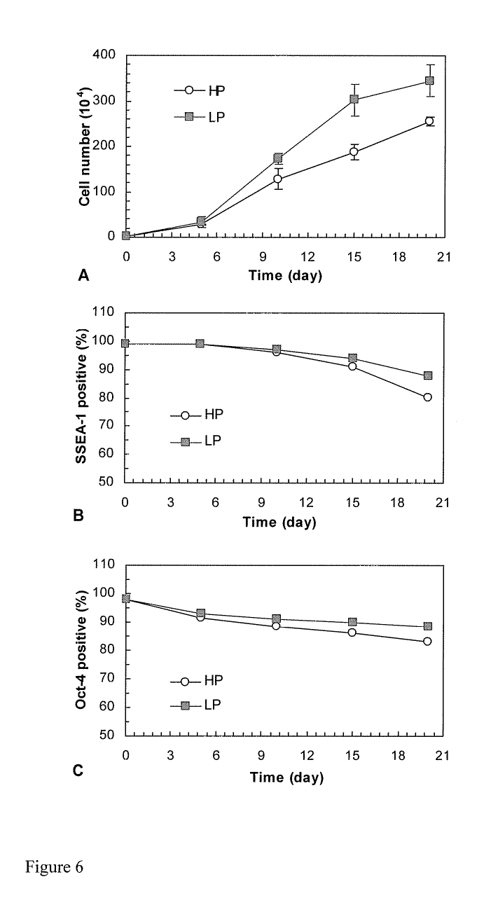

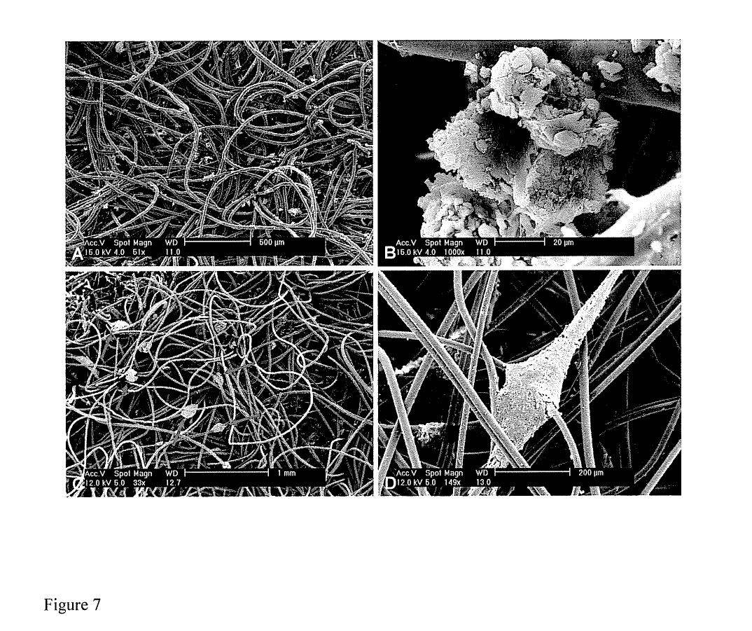

ES cells were cultured in PET matrices with two different pore sizes. As shown in FIG. 6, ES cells cultured in the smaller pore matrix (LP) grew faster with 60% more cells after 20 days as compared to the HP culture. There were also more SSEA-1 and Oct-4 positive cells in the LP culture. With smaller pores, the contact probability between the cells and the fibers is higher. Accordingly, more attachment and bridging, which are beneficial for cell proliferation and maintenance of undifferentiated state, are likely to occur. This pore size effect was confirmed by SEM images showing that ES cells were more likely to form large aggregates in large-pore matrices (HP), and on the other hand more evenly spreading in the entire fibrous matrix when cultured in small-pore matrices (LP) (see FIG. 7). Aggregation of cells, which was mostly found in large-pore matrices, is not conducive to proliferation and can induce spontaneous differentiation caused by contact inhibition. These findings are consistent with the results previously reported and can explain why cells grown in the larger pore matrices were more likely to differentiate.

As also can be seen in the SEM pictures shown in FIG. 7, 3-D PET matrices provided plenty of fiber surface and void space for cell anchorage and growth. There were still tremendous amounts of unoccupied surface and space in the PET scaffolds even in the long-term cultures that reached a relatively high cell density of ˜10⁸ cells/mL matrix volume. It is thus unlikely for the 3-D culture to have contact inhibition that is commonly occurring to 2-D cultures.

Effects of Oxygen Transfer in 3-D Fibrous Matrix

Nutrient limitation in the media, another common cause for cell growth inhibition, was believed to be the main reason for cell growth stagnation in the long-term 3-D cultures observed in this study. In a 3-D static culture, the main mass transfer mechanism is diffusion, which is inefficient for oxygen with a low solubility in water. Oxygen must diffuse through the liquid that occupied the pores of the fibrous matrix in order to reach the cells in the scaffold. When the cell density in the matrix increased as a result of cell growth, the oxygen consumption rate also increased while the permeability of the matrix decreased, resulting in poor oxygen transfer in the scaffold and oxygen starvation.

To verify this oxygen starvation hypothesis, a diffusion model shown below was used to simulate the oxygen concentration changes during the period of long-term 3-D culturing in the PET matrices.

For diffusion in the liquid phase (z≦0.415 cm),

$$\frac{\partial C}{\partial t} = {D}_{1} \frac{{\partial }^{2} C}{\partial {z}^{2}}$$

For diffusion in the PET matrix (0.415 cm<z≦0.515 cm),

$$\frac{\partial C}{\partial t} = {D}_{2} \frac{{\partial }^{2} C}{\partial {z}^{2}} - {QX}_{0} exp ( μ t )$$

The boundary and initial conditions are as follows:

C=0.212 mM at z=0 (oxygen saturation at the surface of the medium)

$$\frac{\partial C}{\partial z} = 0$$

at z=0.515 cm (no diffusion at the well bottom)

C=0.212 mM at t=72n h (n=0, 1, 2, 3, 4, 5, since media were refreshed every 72 h)

The dissolved oxygen concentrations can be found by solving the above transient partial differential equations using Matlab® and the parameter values as follows: oxygen diffusivity in the liquid, D₁=2.5×10⁻⁵ cm²/s and inside the matrix, D₂=1.8×10⁻⁵ cm²/s, the specific oxygen consumption rate, Q=1×10⁻¹⁰ mmol/cell·h, the initial cell number, X₀=3.75×10⁴, the specific growth rate, μ=0.012 h⁻¹. The model-predicted oxygen concentration profiles along the z-direction or the depth from the medium surface during the culturing period were calculated (data not shown), which showed that the oxygen concentration at the bottom of the culture well reduced to zero at 387 h, which is consistent with the experimental observation that cell growth stopped at ˜15 days of culturing (FIG. 5A). It is thus concluded that oxygen depletion was the dictating factor in limiting the long-term 3-D static culture. Better long-term culture performance was obtained when a dynamic culture with better oxygenation was used.

Dynamic Culture in Spinner Flasks

Cell Seeding Kinetics

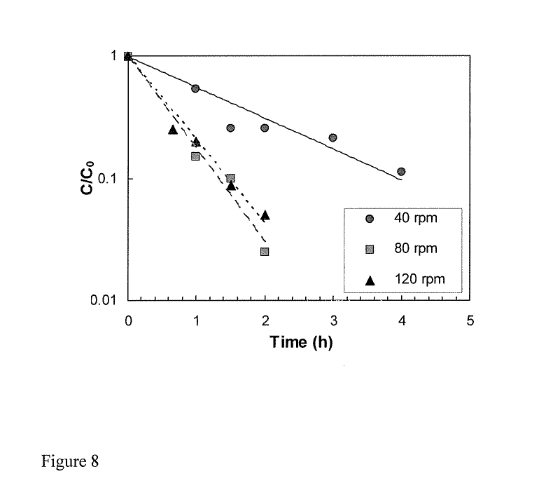

The kinetics of cell seeding in the spinner flask culture was studied by monitoring the number of suspended cells remaining in the culture medium after seeding. The adsorption kinetics of mES cells to the PET matrix followed the first order reaction kinetics, as illustrated in FIG. 8. In addition, the adsorption rate was influenced by the agitation rate, with the adsorption rate constants found to be 0.58 h⁻¹, 1.76 h⁻¹ and 1.58 h⁻¹ at 40 rpm, 80 rpm and 120 rpm, respectively. In all three conditions, the seeding efficiencies were above 90%. Since the contact between cells and fibers was random, increasing the agitation rate increased the contact probability between cells and fibers. However, an overly high agitation rate can also result in an increase in the detachment rate. The optimal agitation rate for cell seeding in the PET matrices was 80 rpm.

Effects of Conditioned Media

Experiments were carried out using conditioned medium to replace the LIF. Results shown in Table 2 imply that there is no significant difference in the cell growth rate among the media studied. However, Oct-4 expression was up-regulated with the increased ratio of the conditioned medium in the culture media; SSEA-1 expression was similar in the three conditioned media. 50% conditioned medium performed as well as LIF containing medium. As expected, mES cells differentiated significantly in the growth medium without LIF, as indicated by their low Oct-4 and SSEA-1 expressions.

TABLE 2
mES cell culture performance after 15 days of dynamic culturing in various media.

| | With LIF | No LIF | 10% CM | 25% CM | 50% CM |
|---|---|---|---|---|---|
| Expansion fold | 128.4 ± 5.3 | 123.8 ± 3.8 | 104.7 ± 4.5 | 118.1 ± 4.5 | 126.1 ± 16.1 |
| Oct-4 (%) | 93.1 | 80.0 | 84.2 | 87.5 | 91.2 |
| SSEA-1 (%) | 91.4 | 76.7 | 87.3 | 87.6 | 88.1 |
| Y_{lac/glu} | 0.63 ± 0.04 | 0.70 ± 0.06 | 0.66 ± 0.04 | 0.51 ± 0.09 | 0.67 ± 0.03 |

In general, the dynamic cultures in spinner flasks performed better than static cultures because of agitation resulting in better oxygen transfer. ES cell expansion was higher in spinner flasks than in static cultures. Furthermore, Oct-4 and SSEA-1 expressions were also higher in the dynamic cultures. The increase in cell expansion and cellular markers was conjectured to be partly caused by the significant decrease in lactic acid production. Since most of lactic acid is produced anaerobically, the production of lactic acid was suppressed by increased oxygen transfer in the dynamic cultures. This was further proven by the lower Y_{lac/glu} values in the dynamic cultures. Consequently, lactic acid concentrations were kept lower than the inhibiting level for most of the time, except for the 50% conditioned media culture.

Effects of Agitation Rate

mES cell cultures under different agitation rates were also compared. The results shown in Table 3 confirmed that increasing the mixing intensity improved mES cell expansion and reduced spontaneous differentiation. When mixing intensity was increased from 80 rpm to 120 rpm, both cell density and Oct-4 and SSEA-1 expressions increased, indicating that mES cell cultures in the 3-D system were mass transfer limited. Mixing provides fresh gas/liquid contact surfaces, and thus more oxygen can be delivered from the gas phase to the media. Also, the mass transfer mechanism involved diffusion as well as convection, which is more effective than diffusion that was the only mechanism in static cultures. The improved mass transfer was needed to ensure that cells in the center of the 3-D matrix did not undergo nutrient (oxygen) starvation.

TABLE 3
mES cell culture performance after 15 days of culturing
in spinner flasks and a two-stage perfusion bioreactor.

| | Cell* | Oct-4 | SSEA-1 | |
|---|---|---|---|---|
| | (10⁶ cells) | (%) | (%) | Y_{lac/glu} |
| Spinner flask | | | | |
| 80 rpm | 59.9 ± 7.6 | 91.2 | 88.1 | 0.63 |
| 120 rpm | 73.6 ± 13.6 | 92.4 | 91.2 | 0.58 |
| Perfusion bioreactor | 1406.7 | 94.6 | 92.4 | 0.55 |

*initial 0.5 million cells were inoculated into the spinner flask with LIF containing medium. Inoculum for perfusion reactor was 7.2 million and no addition of LIF to the reactor

Perfusion Culture in Controlled Bioreactor

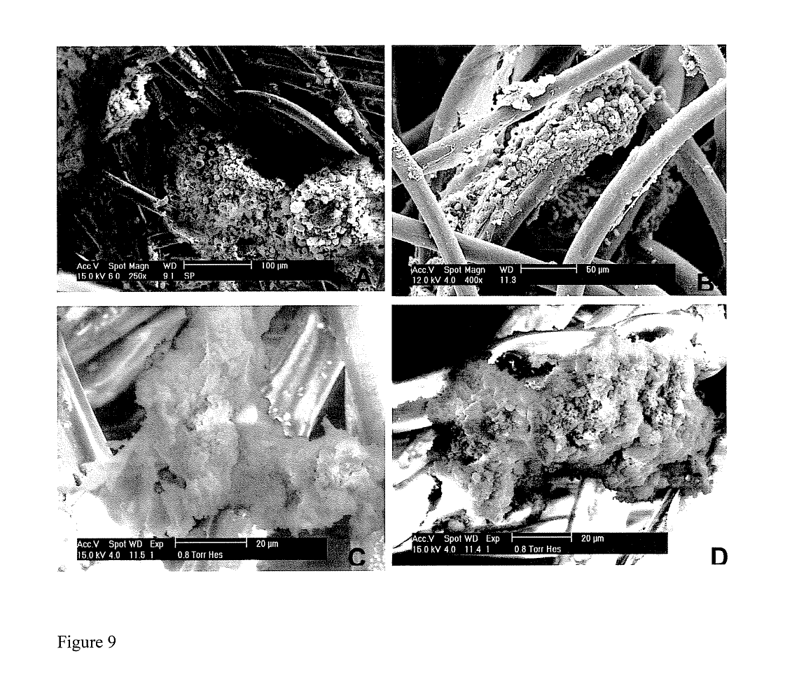

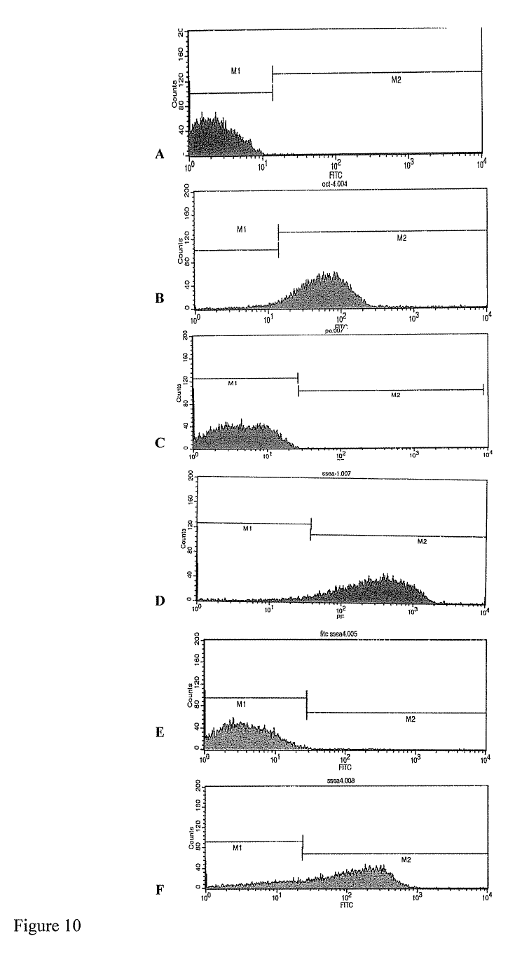

In the previous results, we have shown that the undifferentiated mES cells could grow well in 3-D PET scaffolds when cultured in conditioned media obtained from STO cells. However, as our ultimate objective is to mass-produce ES cells, further up-scaling optimization needed to be performed. For this purpose, a two-stage perfusion culture system shown in FIG. 1 was developed. The final cell number and SSEA-1 and Oct-4 positive cell fractions are given in Table 3. In 15 days, mES cells in this perfusion bioreactor system expanded 193-fold to 1.4 billion cells in the FBB bioreactor, which was much higher than those attained in spinner-flask. High density mES cells morphology in the reactor is shown in FIG. 9A, B. Last but not least, the Oct-4 and SSEA-1 marker expression levels were 94.6% and 92.4% (FIG. 10B, D), respectively, higher than those of the mES cells cultured in spinner flasks and multiwells using LIF-containing growth medium.

By controlling pH and DO as well as proper perfusion, the cell culture environment was optimized in the bioreactor. Since both STO and mES cells were immobilized in the PET matrices and no viable cells were circulating in the culture media, there was almost no cross contamination between the two bioreactors. Furthermore, with the continuous supply of conditioned media from STO cells in the first bioreactor, the majority of the mES cells could conserve their pluripotency, as proved by the high percentage of undifferentiated mES cells. At the end of the study, ES cells in the bioreactor were harvested by rinsing with Accutase, which gave a 47.6% recovery rate and 92% viability.

Human ES Cell Cultures

hES cells culture in spinner flask was also investigated and the results are summarized in Table 4. A great advantage of 3-D culture of hES cells is eliminating the necessity of the Matrigel coating though hES cell growth slowed down to some extent in the 3-D spinner system. Nevertheless, this problem was solved in the bioreactor system. Matrigel is a complex mixture of mouse sarcoma origin. Therefore, animal components still exist in the culture system and this is not desirable for clinical therapy. Although human laminin can replace the Matrigel in hES cells culture, it still imposes a high cost to the process.

TABLE 4
Comparison of hES cell cultures in different systems.*

| | | | SSEA-4 | SSEA-4 |
|---|---|---|---|---|
| | Expansion | Doubling | Initial | Final |
| | Fold | Time (h) | (%) | (%) |
| 2-D cultures (7 days) | 5.7 | 66 | 85.2 | 82.1 |
| Spinner flask (14 days) | 25 | 72 | 85.2 | 79.2 |
| Bioreactor (18 days) | 145 | 60 | 83.2 | 79.4 |

*0.5 million hES cells were cultivated on Matrigel coated 25 cm² T-flask with MEF conditioned medium supplemented with 4 ng/ml bFGF in 2-D culture. 0.3 million hES cells were inoculated into a 25 ml spinner flask with 10 ml working volume packed with 10 × 1 cm PET scaffold.

Human ES cells culture in the perfusion bioreactor demonstrated the success. hES cells expanded 145-fold and the SSEA-4 expression dropped slightly from 83.2% to 79.4% (FIG. 10F) in 18 days, indicating that most of hES cells in the bioreactor maintained their pluripotency during the in vitro expansion. Furthermore, the hES cell growth rate in the bioreactor also surpassed that of the 2-D culture. In summary, no need for the ECM coating, higher growth rate, good maintenance of pluripotency, and automatic control assure the perfusion fibrous bed bioreactor system is a very promising platform for the scale up of the mass production process of undifferentiated hES cells. FIG. 9C, D show hES cells morphology in PET scaffold.

Mathematical Model

To better understand the mass transfer in ordered disk packed FBB, where ES cells grow, an axial dispersion model with a reaction (oxygen consumption) term was developed to predict oxygen consumption and cell growth. The model and the initial and boundary conditions are given as follows.

$$\begin{matrix}\frac{\partial c}{\partial t} = {D}_{l} \frac{{\partial }^{2} c}{\partial {z}^{2}} - \frac{u}{ɛ} \frac{\partial c}{\partial z} - {Q}_{0} {X}_{0} {ⅇ}^{{μ}^{′} \cdot t} & ( 1 ) \\ \begin{matrix}t = 0 , & c ( z , 0 ) = 0\end{matrix} & ( 2 ) \\ \begin{matrix}z = 0 , & c = {c}_{0} + \frac{{D}_{l} ɛ}{u} \frac{\partial c}{\partial z}\end{matrix} & ( 3 ) \\ \begin{matrix}z = L , & \frac{\partial c}{\partial z} = 0\end{matrix} & ( 4 )\end{matrix}$$

In this model, the cell growth was assumed to follow Monad equation

$$μ = {μ}_{max} \frac{C}{K + C} \qquad ( 5 )$$

Where Q₀=10⁻¹⁰ mmol/cell/h [27], μₘₐₓ=0.0146 h⁻¹, X₀=5×10⁵ cell/mL bed volume (from our own experimental data) and K is assumed to be 5% DO.

In order to use the proposed model, the dispersion coefficient, Dₗ, in eq. 2 must be determined from the residence time distribution, E(t). The mean residence time, tₘ, of the fluid in the packed column and its variance, σ_{θ}, can be determined from the following equations.

$$\begin{matrix}E ( t ) = \frac{C ( t )}{{\int }_{0}^{∞} C ( t ) ⅆ t} & ( 6 ) \\ {t}_{m} = {\int }_{0}^{∞} t E ( t ) ⅆ t & ( 7 ) \\ {σ}^{2} = {\int }_{0}^{∞} {( t - {t}_{m} )}^{2} E ( t ) ⅆ t & ( 8 ) \\ {σ}_{θ}^{2} = \frac{{σ}^{2}}{{t}_{m}^{2}} & ( 9 )\end{matrix}$$

where the dimensionless variance, σ_{θ}, is related to the axial dispersion coefficient, Dₗ, or the Bodenstein number,

$$Bo = \frac{uL}{ɛ {D}_{l}} ,$$

as follows:

$${σ}_{θ}^{2} = \frac{2}{Bo} - \frac{2}{{Bo}^{2}} ( 1 - {ⅇ}^{- Bo} ) \qquad ( 10 )$$

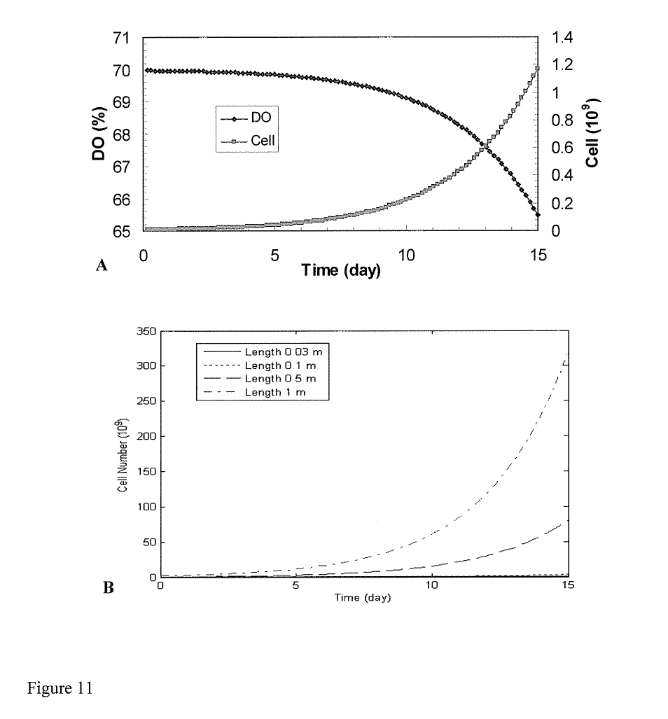

To determine the residence time distribution and the axial dispersion coefficient in the packed column, 0.2 ml of saturated NaCl solution was instantaneously injected into the feed stream into the affinity column. The pulse input method was used to ascertain the mean residence time and variance. The conductivity of the effluent was measured every 5 seconds until the reading returned to its initial value and remained stable for 30 seconds. The data were used to determine the mean residence time, variance, and then the dispersion coefficient of salt in the affinity column, which was then used to estimate the dispersion coefficient of oxygen on that Dₗ is proportional to diffusivity D_{eff}. D_{eff} of NaCl is 1.6×10⁻⁵ cm²/s and that value for oxygen is 2.5×10⁻⁵ cm²/s. Oxygen at the outlet of the reactor and total cell number in the reactor were shown in FIG. 11A. The model illustrated that DO was above 60% by continuous aeration during the culture and cells were evenly distributed along the reactor. The model also predicted that ˜1.2 billion ES cells could be obtained after 15 days This value is comparable to our experimental data (1.4 billion cells), indicating the accuracy of the model. The mathematical model can be used for predicting FBB performance under various operation conditions and thus providing invaluable information for process scale up. As such, the reactor was up-scaled to larger size and cell growth was predicted as shown in FIG. 11B.

Discussion

ES cells are recognized as a perfect cell source for cell therapy because of their unlimited proliferation potential and pluripotency. Large quantity of cells is then required in cell therapy. For instance, 30 million hematopoietic cells/kg of the patient will be transplanted in the leukemia treatment. A normal weighted adult patient (70 kg) will need 2.1 billion cells in one treatment. Suppose the differentiation efficiency from ES cells is 30%, then 7 billion undifferentiated ES cells are needed for one treatment. If using current 2-D culture method in ECM coated T-flask, 2000 T-flasks (75 cm²) will be needed to produce these cells.

Cell or tissue transplantation is also employed in Parkinson's Disease (PD) treatment. Human fetal esencephalic tissue, rich in dopaminergic neurons, was first transplanted to a PD patient in 1987. However, several fetuses required even for one patient. 20,000 dopamine neurons are required for mouse transplantation and approximately 20 million dopamine neurons would be required for an adult PD patient (estimation according to animal model data). The efficiency of hES cell differentiation into dopamine neuron (tyrosine hydroxylase (TH)⁺ cells) was low (˜1%). Therefore, approximately 2 billion ES cells are required for one PD patient, which will need ˜1000 T-flasks (75 cm²).

Cell therapy also found the application in diabetes treatment. Up to 2˜4×10⁹ β-cells are involved in current transplantation protocols. The ES cell differentiation efficiency into β-cells is around 15%. 13˜26 billion ES cells will be required for an adult diabetes patient, which will use 4000˜8000 T-flasks (75 cm²). The ECM protein cost and labor cost involved in thousands of T-flask subculture impose more obstacles for the affordability of cell therapy. Furthermore, too many human involvements in the process make it hard to control the consistency of the product, ES cells in this case. It is obvious that an economical and automatically controlled bioreactor process development is one of the priorities in ES cell therapy study. However, the large scale production of undifferentiated ES cells is rarely reported due to its anchorage dependency and high expense of growth factors. One possible scalable way using microcarriers has been reported for mass production of the ES cells.

The two-stage perfusion FBB system overcomes the shortcomings in current ES cell expansion process. Huge surface area in PET scaffold prevents cell contact inhibition in 2-D system. Continuous perfusing STO conditioned medium as well as pH and DO control optimize ES cells growth environment. ES cells keep growing in the PET scaffold and maintain their pluripotency without expensive growth factor LIF and subculture. 1.4 billion cells were obtained in the FBB in 15 days at current inoculum. By increasing the current reactor volume four times or extend the culture time to 21 days (model prediction), 7 billion cells required for one leukemia treatment can be easily obtained. Similarly, the ES cells required for one PD treatment and diabetes treatment can be obtained within a month using the FBB as illustrated in this work. The process is also automatically controlled, making the process more friendly and robust. In order to meet the timelines of certain clinical trials or shorten the period of waiting time for the cells, large scale FBB system should be employed. Suppose 1×10⁴ ES cells/ml cells were cultivated in a 10 L bioreactor with 2 L packing materials. 1.2 billion cells can be obtained after a week and 13.7 billion cells after 2 weeks. 159 billion cells can be obtained after 3 weeks and these cells are enough for 20 leukemia patients, or 80 PD patients or 10 diabetes patients. Cell density at this time is 7.95×10⁷ cells/ml matrix, which is achievable and lower than the highest cell density obtained in FBB (3×10⁸ cells/ml matrix). Some patients on the waiting list for tissue or cell transplantation have to wait for more than a year due to the shortage of the donors. Mass production of ES cells in FBB makes it possible to treat those patients promptly.

hES cells expansion is more complicated. They were usually cultivated on Matrigel or laminin in MEF conditioned medium, where they could maintain their pluripotency for more than 130 population doublings. It has been found that clonally derived human embryonic stem cell lines could maintain pluripotency and proliferation potential for prolonged periods of time. It has also been shown that hES cells can be maintained on a fibronectin matrix using unconditioned medium by supplementing bFGF and TGF-β1. Nevertheless, hES cell showed lower cloning efficiencies and growth rates and higher possibility of spontaneous differentiation. It has been observed that human adult marrow cells support prolonged hES cell expansion. It has been shown that bFGF alone or with other growth factors supports hES cell growth which is comparable to the hES cells growth in MEF conditioned medium. It has been ascertained that high concentration of bFGF (250 ng/ml) was efficient to support undifferentiated hES cells growth. It has been reported that 50 ng/ml of activin A was capable of sustaining hES cells pluripotency over 20 passages without the feeder layer or MEF conditioned medium. Without the presence of bFGF, hES cell's pluripotency can be maintained in unconditioned medium by supplementing TGF-β or activin, but the cell growth is poor.

In the present perfusion FBB system for hES cells culture, no other growth factors except 4 ng/ml bFGF was included in the culture medium, which is significantly lower than the published data (250 ng/ml bFGF), indicating another advantage of the FBB system.

### CONCLUSIONS

In summary, The 3-D culture system is an easy and economical way to mass produce undifferentiated ES cells. ECM coating and frequent subculture were circumvented in 3-D ES cell cultures. Lactic acid above a certain threshold has a negative effect on ES cell growth and initiates differentiation. ES cells attachment on PET scaffold follows the first order reaction. ES cells growing in the perfusion fibrous bed bioreactor system kept satisfactory growth rate and maintained their pluripotency. The success of two-stage perfusion fibrous bed bioreactor system demonstrates the promising future for up-scaling the process to satisfy market demands. An axial dispersion model was developed to simulate the mass transfer and cell growth inside the reactor. The predicted cell number and actual cell number are comparable, indicating the accuracy of the model.

### EXAMPLE 2

### Neural Differentiation from Embryonic Stem Cells in Different Culture Systems

Materials and Methods

Cultures and Media

ES D3 cells (CRL-1934) obtained from ATCC were maintained on gelatin pre-coated T-flasks consisting knock-out Dulbecco's Modified Eagle's Medium (DMEM) supplemented with 10% fetal bovine serum (FBS), 50 U/mL penicillin, 50 μg/mL streptomycin, 0.1 mM non-essential amino acids, 2 mM L-glutamine, 100 μM β-mercaptoethanol (Sigma) and 100 μM leukemia inhibitory factor (LIF) (Chemicon). For neural differentiation, LIF was excluded in the above ES medium and either retinoic acid (10⁻⁷ M) or an astrocyte-conditioned medium (30% v/v) was added to induce neural differentiation.

Astrocytes (CRL-2253) from ATCC were cultured in DMEM with 10% FBS. Approximately three to five million astrocyte cells were inoculated to a PET matrix submerged in 110 mL of DMEM with 10% FBS in a 250-mL spinner flask. The medium was refreshed at day six and cells were cultured for another four days in the medium with 10% knockout serum replacement instead of serum. Supernatant was then collected and used as conditioned medium to mix with fresh ES cell medium without LIF for neural differentiation.

Embryoid Body (EB) Formation and Re-Inoculation

To produce the EB body for neural differentiation study, ˜2-5×10⁵ ES cells were inoculated into the 9-cm bacterial culture grade Petri dish containing 10 ml of the differentiation medium. After 4 days of culturing, 9 ml of supernatant was discarded and EBs were harvested and suspended in 1 ml medium and used in neural differentiation study in various culturing systems described below. The total number of cells in the EB suspension was estimated by nuclei counting of cells present in 100 μl of EB suspension.

Nonwoven PET Matrices

Needle-punched nonwoven polyethylene terephthalate (PET) matrices (fiber diameter, ˜20 μm; fiber density, 1.35 g/cm³) were used as cell culture scaffolds. Pre-treatment of the PET matrices was necessary prior to use in order to increase the hydrophilicity. 1% (w/v) Na₂CO₃ and 1% (v/v) Tween-20 were mixed together and heated to 60° C. PET matrices were cut into required shape and kept in the above solution for 30-60 min. After being rinsed with distilled water several times, the PET matrices were transferred to 1% (w/v) NaOH solution and kept boiling for 30-60 min. Finally, matrices were thoroughly washed with distilled water several times.

Neural Differentiation of ES Cells in Static Cultures

In static cultures, EBs were inoculated into either gelatin coated 24 well plate (2-D culture) or 3-D PET matrices in 24 well plate. Each well was inoculated with EBs equivalent to ˜0.1 million cells (2-D culture). In 3-D inoculation, 50 μl of EBs suspension equivalent to ˜0.12 million cells were slowly added to the matrix from the center by pipette. After incubation in a CO₂ incubator for 3 h to allow for cell attachment to the matrix, the seeded matrix was washed with medium to remove unattached and loosely attached cells. Then, the matrix was transferred to a new well and 1 ml of fresh medium was added.

Dynamic Cultures in Spinner Flasks

The EBs were inoculated and cultured in 25-ml spinner flasks with 10 ml of conditioned media and 2 mg/ml of microcarriers (Cytodex-3, Pharmacia, Uppsala, Sweden) or a piece of PET fibrous matrix (10 cm×1 cm×0.18 cm), representing 2-D and 3-D culturing environments, respectively. Spinner flasks were pre-siliconized with Sigmacote® (Sigma) to eliminate the possibility of cell attachment to the flask wall. The PET matrix was affixed on a stainless steel mesh around the wall of the spinner flask. After autoclaving at 121° C. for 30 min, 10 ml of EB suspension (equivalent to ˜1 million cells) were added to the spinner flask. For the 2-D dynamic culture, microcarriers were pre-hydrated with PBS for 5 h, autoclaved at 121° C. for 30 min, and then soaked with the culture medium overnight prior to seeding with 10 ml of EB suspension (equivalent to ˜1 million cells). Unless otherwise noted, the spinner flasks were incubated in a 5% CO₂ incubator at 37° C., agitated at 80 rpm, and with media change once every 2-3 days.

De-Differentiation of Partially Differentiated ES Cells

The partially differentiated ES cells from 2-D cultures in the astrocyte conditioned medium were harvested after 7 days and then re-inoculated into ES cell growth medium, with or without LIF, and the differentiation medium (30% v/v conditioned medium plus 10⁻⁷ M retinoic acid) in both 2-D and 3-D cultures to study the potential of de-differentiation of ES cells under various culturing conditions.

Flow Cytometric Analyses

To identify neural differentiation, two intracellular proteins, nestin and NCAM were analyzed. Nestin is expressed in the early stage of neural differentiation; while NCAM is expressed in the late stage. Undifferentiated ES cell marker Oct-4 was also analyzed. ES cells were trypsinized from the solid supports (2-D surface, 3-D PET matrices, and microcarriers) prior to the flow cytometric analysis. To measure the intracellular proteins Oct-4, nestin, and NCAM, cells were fixed with 4% formaldehyde for 20 min. at room temperature, and then the cell membranes were perforated with the permeabilizing buffer containing 0.5% saponin and 0.1% sodium azide (Sigma, St. Louis, Mo.) for another 20 min at room temperature. The samples were incubated with anti-Oct-4 (Chemicon, Temecula, Calif.), anti-nestin, or anti-NCAM monoclonal antibody (Developmental studies hybridoma bank, Iowa city, Iowa) at room temperature for 30 min. After washing with the permeabilizing buffer, cells were incubated with IgG-FITC (Jackson Immuno Lab, West Grove, Pa.) for another 30 min. Finally, cells were fixed with 2% formaldehyde and analyzed with a flow cytometer (BD FACS Calibur). Unstained cells were used for locating the population and cells only labeled with IgG-FITC were used to evaluate the non-specific binding or background fluorescence reading. At least 10,000 gated cells out of the whole cell populations (more than 10,000 cells) were measured by flowcytometer for each marker. Therefore, the method itself is statistically significant without replication.

Cell Counting in 3-D PET Matrices

The PET matrix was put into the standard nuclei counting solution (0.1 M citric acid, 0.1% (w/v) crystal violet) and incubated at 37° C. for 24 hours. The matrix was strongly vortexed prior to counting.

Scanning Electron Microscopy

The 0.5×0.5 cm² samples were fixed with 2.5% glutaraldehyde overnight at 4° C. After washing with PBS several times, samples were dehydrated for 30 min in each of the ethanol solutions with ascending concentrations: 20%, 30%, 40%, 50%, 60%, 70%, 80%, 90%, and 100%. Samples were then transferred to hexamethyldisilazane (HMDS) (Sigma) and ethanol mixtures with ascending HMDS concentrations: 1:3, 1:1, and 3:1 for final dehydration. Samples were coated with gold/palladium and examined with a Philips XL 30 scanning electron microscope (SEM) (Philips Electronics, Eindhoven, The Netherlands).

Results

Effect of Conditioned Medium on Neural Differentiation

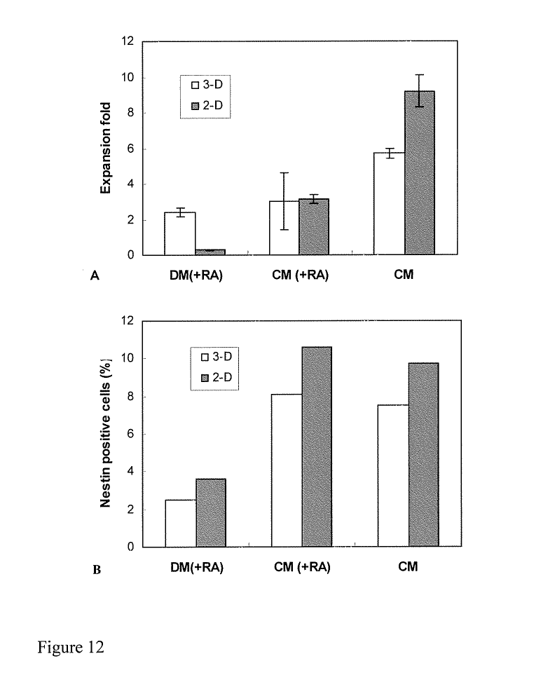

It has been reported that 10⁻⁷ M of retinoic acid (RA) can induce neural differentiation in ES cells. However, it was found that retinoic acid treatment strongly inhibited cell proliferation in both 2-D and 3-D cultures (FIG. 12A). Cells in astrocyte-conditioned medium grew ten times faster than cells in retinoic acid containing medium. Cells cultured in the conditioned medium with retinoic acid also grew faster than those in retinoic acid medium. The ability of conditioned medium to induce neural differentiation was studied and it was found that the conditioned medium was more efficient than retinoic acid in terms of neural differentiation induction (FIG. 12B). Nestin expression in the conditioned medium was 3 times higher than that in the medium containing retinoic acid. Although conditioned medium plus retinoic acid had the highest nestin expression, conditioned medium by itself was better for neural differentiation because of its better performance in both proliferation and differentiation. It is noted that cells in 2-D cultures generally had higher nestin expressions, suggesting that neural differentiation was more efficient in 2-D cultures. This was further studied with additional neural differentiation marker NCAM discussed below.

Neural Differentiation in Static Cultures

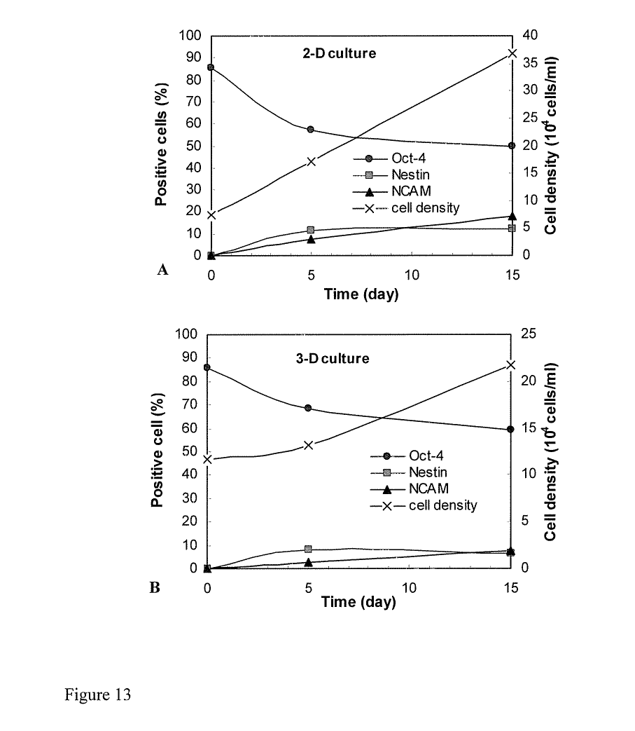

FIG. 13 shows the kinetics of neural differentiation of ES cells grown either on 2-D surfaces or in 3-D matrices under static culture conditions. In general, ES cells cultured in these systems proliferated and gradually differentiated into neural cells as indicated by the increasing cell populations expressing nestin and NCAM and decreasing cell population expressing Oct-4 during the 15-day culturing period studied. However, both proliferation and neural differentiation were faster for ES cells grown on the 2-D surface in Petri dishes than they did in 3-D PET matrices.

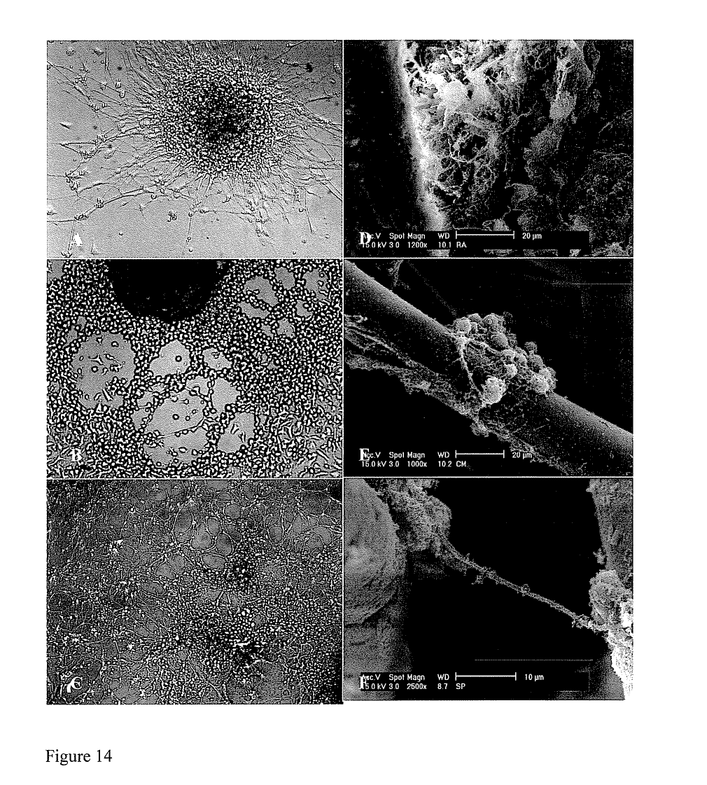

FIG. 14 shows the cell morphology in 2-D and 3-D culture systems. For 2-D cultures, cells in the periphery of the attached embryoid body differentiated into neural type cells first (FIG. 14A). These periphery cells also migrated out and communicated with other attached embryoid bodies if there was one nearby by forming neurites or bridges (FIG. 14B) and eventually created a neural network (FIG. 14C). For 3-D cultures in PET fibrous matrices, cells can form sophisticated neural networks in high cell density regions (FIG. 14D). They stretched out on the fiber as they did on the 2-D surface (FIG. 14E). Two cells on the adjacent fibers could also communicate with each other by forming the bridge (FIG. 14F).

Neural Differentiation in Dynamic Cultures

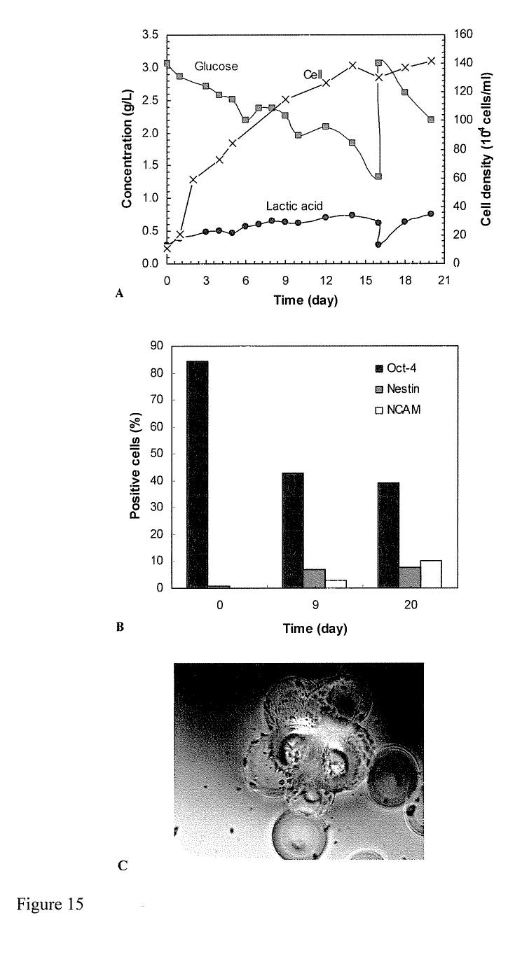

Dynamic culturing in a bioreactor with mixing can improve mass transfer and afford a more scaleable cell culture process, and was thus studied for its use in neural differentiation of ES cells. FIG. 15 shows neural differentiation of ES cells in the 2-D microcarrier culture system for a period of 21 days. In general, the ES cells in the dynamic culture continued to grow and expanded ˜14-fold (FIG. 15A). During this period, Oct-4 was increasingly down-regulated, while both neural markers, nestin and NCAM were up-regulated (FIG. 15B). The metabolic rates, as indicated by the glucose and lactic acid concentrations in the culture medium (FIG. 15A), remained stable, indicating a good potential for long-term culture in this system. However, large aggregates of embryoid bodies and multiple microcarriers (see FIG. 15C) were found in this system. The formation of large EB-microcarrier aggregates lowered the efficiency of utilizing the surface areas provided by the microcarriers and might have slowed neural differentiation as compared to the static culture in multiwells.

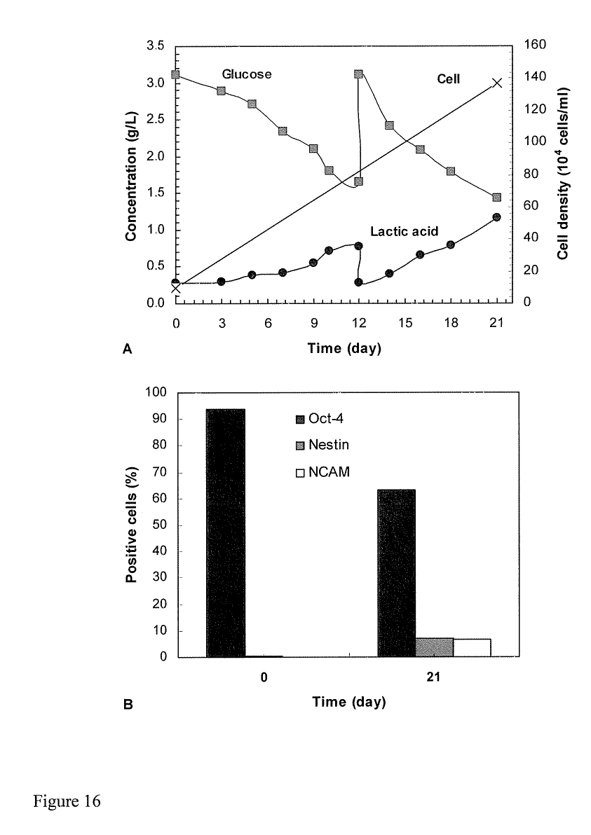

Neural differentiation in 3-D dynamic culture in the spinner flask with the PET fibrous matrix as the cell support is shown in FIG. 16. Compared to the 2-D microcarrier culture system, the 3-D culture had a similar growth rate, but a lower efficiency in inducing neural differentiation as indicated by the higher Oct-4 and lower nestin and NCAM expressions at 21 days (FIG. 16B). This finding was consistent with the results from the static cultures discussed before.

It should be noted that the degree of ES cell neural differentiation in these dynamic cultures was lower than those obtained in static cultures, indicating that the dynamic cultures were not optimal for neural differentiation. The reasons for this will be discussed later.

De-Differentiation of Partially Differentiated ES Cells

The de-differentiation of partially differentiated ES cells were studied in both 2-D and 3-D static cultures and the results are shown in Table 5. In general, partially differentiated ES cells continued their differentiation process when they were re-cultured in the differentiation medium, as indicated by the increased expressions of nestin or NCAM after 14-day culturing. However, the differentiation process was interrupted for cells re-cultured in the two growth media, either with or without LIF. As expected, cells cultured in these media showed a much higher growth rate than that in the differentiation medium. Furthermore, significant increase in Oct-4 (from 48% to 60˜70% in 2-D and 70˜80% in 3-D cultures) and decreases in both nestin and NCAM expressions were observed in these cultures at 14 days. This population shift toward undifferentiated cells might be because the non-differentiated ES cells in the mixed population grew faster than the differentiated cells. However, it is also possible that “de-differentiation” occurred when partially differentiated cells were re-cultured in the growth media. This hypothesis was made with more confidence based on the observation that the cell growth rate in 3-D cultures was usually less than that in 2-D cultures. The observation that slower growing cells (in 3-D) had higher Oct-4 expression and lower Nestin/NCAM expression can be attributed to the de-differentiation. The 3-D culture clearly is more favorable for the proliferation of undifferentiated ES cells than 2-D culture and has the higher potential to induce de-differentiation under the culturing conditions studied.

TABLE 5
De-differentiation of partially differentiated ES cells
in various media in 2-D and 3-D static cultures.

| | Cell density | Oct-4 | Nestin | NCAM |
|---|---|---|---|---|
| | (10⁴/well) | (%) | (%) | (%) |
| Initial cell population | ˜10 | 48 | 13 | 7 |
| After 2-D culturing in: | | | | |
| Growth medium with LIF | 70 ± 15 | 69 | 20 | 2 |
| Growth medium without LIF | 73 ± 29 | 60 | 20 | 4 |
| Differentiation medium | 25 ± 14 | 50 | 4 | 31 |
| After 3-D culturing in: | | | | |
| Growth medium with LIF | 33 ± 3.1 | 80 | 9 | 0.1 |
| Growth medium without LIF | 66 ± 7.5 | 78 | 9 | 2 |
| Differentiation medium | 5 ± 0.5^{a} | 60 | 18 | 5 |

^{a}The cell number was lower than the total cell number used to inoculate the PET matrix because not all cells were attached to the matrix in the initial cell seeding.

Discussion

10⁻⁷ M of retinoic acid has been applied to induce neuron differentiation of ES cells. After 4-5 days of treatment, neuron marker GAP-43 and NF-165 expression was detected. However, neurogenesis is a multifaceted process—different neural cells with different markers appear at different stages. A panel of neural markers such as nestin, sox1 RC2, β-tubulin III, glial fibrillary protein (GFAP), NCAM were detected for different stage of neural cells. ES cells are capable of differentiating into neural stem cells, which can further differentiate into astrocyte progenitor, oligodendrocyte progenitor, and neuronal progenitor cells. These progenitor cells finally differentiate into more specific neural cell types. Neural progenitors derived from ES cells have been studied. In order to obtain enriched neuron progenitor cells, ES cells were cultured for 3-4 weeks, and during the first week, neuronectodermal markers nestin and PAX-6 were detected. After one week, the differentiation process speeds up, and early neuronectodermal marker NCAM (neural cell adhesion molecule) was expressed. They also found that combining basic fibroblast growth factor (bFGF) and epidermal growth factor (EGF) is favorable for human fetal and adult derived neuroepithelia progenitors.

Dopaminergic neurons attracted particular attention due to their application in Parkinson disease treatment. Two different procedures to derive dopaminergic neurons from ES cells have been reported. One is based on defined culture conditions and the other is based on co-culturing with stromal feeder cells. One developed a method for generating midbrain neurons in a defined culture medium; the whole process took 3-5 weeks. The other developed a co-culture system to induce midbrain dopaminergic neuron differentiation from mouse ES cells. They identified a stromal cell-derived inducing activity (SDIA) that increased neural differentiation. A similar strategy to derive midbrain dopamine neurons from human embryonic stem cells by using MS5 and S2 as feeder cells has also been used. It has been found that astrocyte-derived factors induced the differentiation of ES cells into neurons. The astrocyte conditioned medium was supplemented with 20 ng/mL fibroblast growth factor-2 (FGF-2) to stimulate the proliferation of neural stem cells. Similar work on hES cells has also been done. In addition, molecular genetic tools to enhance the midbrain dopamine neuron differentiation efficiency have been applied.

Although retinoic acid was effective in inducing neural differentiation, its induction on neural differentiation was dose dependent. It has been reported that cardiac muscle cells were observed when retinoic acid was depleted in the culture medium. Moreover, retinoic acid significantly inhibited cell proliferation. A cocktail of chemicals and growth factors for neural differentiation has been applied, but this method involved expensive growth factors. A combination of FGF2, BMP4, KAAD, SU5402 to initiated the neural differentiation from ES cells has been used. A more delicate but also economical neural differentiation method thus should be developed for future large scale process. Therefore, astrocyte conditioned medium was studied as an alternative method to induce dopamine neurons from ES cells. Our results proved that astrocyte conditioned medium can induce neural differentiation in ES cells more efficiently than retinoic acid. It also has the advantage of stimulating cell growth.

It should be noted that Nestin and NCAM values from flowcytometry are low. Reported quantitative data of the two marker expression were based on the “positive colonies out of total colonies” instead of the individual cells. However, nestin positive cells were on the periphery of the colony and cells in the center were still Oct-4 positive. A nestin positive colony still contained a large quantity of Oct-4 positive cells. Therefore, their reported value was higher (60%-80%) than our data. It has also been found that neural differentiation on the gelatin coated surface was low, which agreed with our observation. It has been discovered that coating condition had a critical effect on the neural differentiation. These findings indicated another engineering way to regulate the ES cell differentiation and the importance of the scaffolding or culture environment on ES cell differentiation.

The significance of interaction between the culture system and cell growth or differentiation has long been recognized. The concept of 3-D cell culturing has been proved to be superior to 2-D culturing in many ways and is widely used in tissue engineering. Three-dimensional tissue scaffolds can mimic cells' in vivo growth environment and thus, cells show their native morphology and functions in 3-D scaffolds. Our previous work also suggested that ES cells were able to maintain their pluripotency and proliferation potential in 3-D PET scaffolds. However, the 3-D culture is not optimal for neural differentiation as compared to traditional 2-D culture systems. Further investigation on this issue revealed an interesting phenomenon of “de-differentiation” in 3-D culture. At least some of the differentiated cells regained their Oct-4 expression ability. Oct-4 is the marker that identifies undifferentiated ES cells. This might be one reason why it worked better for undifferentiated ES cells expansion in 3-D PET scaffolds. However, others found that Oct-4 expression was maintained when neural differentiation was performed with the co-culture with PA6 cells. Anyhow, Oct-4 expression was down-regulated rapidly when neural differentiation was performed on poly-L-ornithine and fibronectin coated dishes. It has been observed that Oct-3/4 expressed in neurospheres composing neural stem cells. It has been ascertained that the down-regulation of Oct3/4 was necessary for neuronal differentiation. However, re-gaining the Oct-4 expression ability from ES derived neural cells has not been reported yet. It has been stated that the down-regulation of Oct 3/4 was clear. But the up-regulation was not significant, if there was. As a result, the significant increase of Oct-4 in this work was because of the de-differentiation.

A dynamic culture system with microcarriers was also found to be better for ES cell neural differentiation than that using 3-D PET scaffolds. Microcarriers provided a 2-D surface area while PET matrices provided a 3-D culture environment. Again, the different differentiation efficiencies can be attributed to de-differentiation in 3-D culture systems.

Compared to static cultures in multiwells, dynamic culturing in bioreactors presents a more scaleable process but was found to be less favorable to ES cell neural differentiation. Mixing provided a better mass transfer rate and thus reduced the accumulation of lactic acid, which is widely considered a cytotoxic metabolic waste. It has been shown that mixing can improve undifferentiated ES cell expansion while still retaining their pluripotency. Bioreactors were also demonstrated to be able to improve the EB forming efficiency. However, others found that mechanical stress could inhibit human embryonic stem cell differentiation. It has been found that embryoid bodies did not grow if put directly into the agitated bioreactor, and they used encapsulation to solve this problem. Others exploited gel capsulation technique to generate cardiomyocyte from ES cells in bioreactors. Therefore, shear stress induced by agitation may contribute to the poor differentiation efficiency, which presents a challenge to the differentiation process scale up in the dynamic culture. Other culture parameters such as pH and DO can also influence stem cells differentiation. It has been found that the differentiation efficiency of erythroid progenitors enhanced when increasing the pH from 6.95 to 7.4. It has been illustrated that 4% oxygen tension promoted more cardiomyocyte differentiation from ES cells than 20% oxygen tension. A scalable neural differentiation process from ES cells in a 3-D bioreactor was demonstrated in this study, which provided a good platform for investigating the effects of these culture parameters. It should be mentioned that ES cells can grow well in the 3-D PET scaffold without pre-coating with any extracellular matrix proteins, a potential cost advantage over 2-D cultures, which usually require coating the tissue flask surface with expensive gelatin or Matrigel. However, more sophisticated process development, control and bioreactor design would be required to optimize ES cell differentiation in the dynamic environment inside a bioreactor.

### CONCLUSION

The astrocyte conditioned medium can induce neural differentiation in ES cells more efficiently than retinoic acid and still stimulate cell proliferation, which provides an economical alternative to expensive cytokines for large scale process. ES cell neural differentiation was more efficient in 2-D than in 3-D cultures. However, sophisticated neural network was observed in 3-D culture. Partially differentiated ES cells can de-differentiated if they were re-cultured in ES cell growth medium with or without LIF. De-differentiation was more significant in 3-D system than in 2-D system. Neural differentiation from ES cells in a 3-D bioreactor is feasible but requires further process optimization.

Other embodiments of the methods and apparatuses will be apparent to those skilled in the art from consideration of the specification and practice of the methods and apparatuses disclosed herein. It is intended that the specification and examples be considered as exemplary only, with a true scope and spirit of the methods and apparatuses being indicated by the following claims.

What is claimed is:

1. A method of culturing stem cells comprising:
growing the stem cells in a first bioreactor comprising a three-dimensional scaffold;
co-culturing in a second bioreactor feeder cells and delivering, either intermittently or continuously, media from the second bioreactor to the first bioreactor; and
perfusing the stem cells with culture medium.

2. The method according to claim 1, wherein the three-dimensional scaffold comprises a non-woven fibrous matrix.

3. The method according to claim 1, wherein the non-woven fibrous matrix comprises a non-woven polyester matrix.

4. The method according to claim 2, wherein the non-woven polyester matrix is polyethylene terephthalate.

5. The method according to claim 1, wherein the non-woven fibrous matrix exhibits average pore size of less than or equal to about 150 μm.

6. The method according to claim 4, wherein the non-woven fibrous matrix exhibits average pore size of from about 20 μm to about 150 μm.

7. The method according to claim 5, wherein the non-woven fibrous matrix exhibits average pore size of from about 30 μm to about 60 μm.

8. The method according to claim 1, wherein the medium comprises feeder cell-conditioned medium.

9. The method according to claim 7, wherein the feeder cells are fibroblast cells.

10. The method according to claim 8, wherein the feeder cell-conditioned medium is prepared by perfusing fibroblast cells with cell culture medium.

11. The method according to claim 9, further comprising growing the fibroblast cells on a three-dimensional matrix.

12. The method according to claim 1, wherein the perfusion is continuous.

13. The method according to claim 7, wherein the culture medium comprises at least one of cytokine leukemia inhibitory factor and other growth factors necessary for stem cell growth.

14. The method according to claim 1, wherein the three-dimensional scaffold comprises at least one protein or ECM coating, wherein the protein or ECM is chosen from gelatin, laminin, fibronectin, collagen, Matrigel, and artificial ECM made of nanofibers.

15. The method according to claim 7, further comprising monitoring the medium for at least one of pH and degree of oxygenation.

16. The method according to claim 14, further comprising adjusting at least one of pH and degree of oxygenation.

17. The method according to claim 9, further comprising filtering the fibroblast feeder cell-conditioned medium before delivering the medium to the first bioreactor.

18. A method of producing a differentiated cell culture, comprising culturing stem cells according to the method of claim 1, wherein the stem cells are co-cultured with cells of the differentiated cell type.

19. A method of producing a differentiated cell culture, comprising culturing stem cells according to the method of claim 1, wherein the stem cells are perfused with medium comprising differentiated cell-conditioned culture medium.

20. A multi-stage bioreactor for growing embryonic stem cells, comprising a first fibrous bed bioreactor in fluid communication with a second fibrous bed bioreactor, wherein the first fibrous bed bioreactor is adapted for growing embryonic stem cells.

21. The multi-stage bioreactor according to claim 19, further comprising a filter separating the first fibrous bed bioreactor from the second fibrous bed bioreactor, and in fluid connection with the first and second fibrous bed bioreactors.

22. The multi-stage bioreactor according to claim 20, further comprising at least one tank in fluid connection with the second fibrous bed bioreactor.

23. The multi-stage bioreactor according to claim 21, wherein the at least one tank is a stem cell seeding tank.

24. The multi-stage bioreactor according to claim 21, wherein the at least one tank is a stem cell harvesting tank.

25. The multi-stage bioreactor according to claim 21, wherein the at least one tank is a waste tank.

26. The multi-stage bioreactor according to claim 21, comprising a stem cell seeding tank, a stem cell harvesting tank, and a waste tank, each in fluid connection with the second fibrous bed bioreactor.

27. The multi-stage bioreactor according to claim 20, further comprising at least one tank in fluid connection with the first fibrous bed bioreactor.

28. The multi-stage bioreactor according to claim 26, wherein the at least one tank is a culture medium reservoir.

29. The multi-stage bioreactor according to claim 19, further comprising at least one control means for monitoring and/or adjusting oxygenation and/or pH of the first and/or second fibrous bed bioreactor.
